(19) United States
(12) Patent Application Publication
(10) Pub. No.: US 2017/0096657 A1
(43) Pub. Date: Apr. 6, 2017

(54) PROCESSES FOR PREPARING SILICA-CARBON ALLOTROPE COMPOSITE MATERIALS AND USING SAME

(71) Applicant: LES INNOVATIONS MATERIUM INC., Granby (CA)

(72) Inventors: Mathilde GOSSELIN, Granby (CA); Ce Guinto GAMYS, Granby (CA); Nadi BRAIDY, Sherbrooke (CA); Jean-Francois LEMAY, Grand-Mere (CA); Kossi E. BERE, Sherbrooke (CA); Charles GAUDREAULT, Waterloo (CA)

(21) Appl. No.: 15/125,376

(22) PCT Filed: Mar. 11, 2015
(86) PCT No.: PCT/CA2015/000155

§ 371 Date: Sep. 12, 2016

Related U.S. Application Data
(60) Provisional application No. 61/951,228, filed on Mar. 11, 2014.

(51) Int. Cl. C12N 11/14; B01J 20/10; C02F 3/10; B01J 20/28; B01J 20/32; B01J 20/20; B01J 20/22

(52) U.S. Cl. CPC C12N 11/14; B01J 20/20; B01J 20/103; B01J 20/22; B01J 20/28004; B01J 20/28021; B01J 20/3204; B01J 20/324; B01J 20/3234; B01J 20/3293; C02F 3/107

(57) ABSTRACT

The present document describes a carbon allotrope-silica composite material comprising a silica microcapsule comprising a silica shell having a thickness of from about 50 nm to about 500 μm, and a plurality of pores, said shell forming a capsule having a diameter from about 0.2 μm to about 1500 and having a density of about 0.001 g/cm3 to about 1.0 g/cm3, wherein said shell comprises from about 0% to about 70% Q3 configuration, and from about 30% to about 100% Q4 configuration, or wherein said shell comprises from about 0% to about 60% T2 configuration and from about 40% to about 100% T3 configuration, or wherein said shell comprises a combination of T and Q configurations thereof, and wherein an exterior surface of said capsule is covered by a functional group; a carbon allotrope attached to said silica microcapsule. Also described is a carbon allotrope-silica composite material comprising a carbon allotrope attached to a silica moiety comprising a silica nanoparticle having a diameter from about 5 nm to about 1000 nm, wherein an exterior surface of said silica nanoparticle is covered by a functional group.

### CROSS-REFERENCE TO RELATED APPLICATIONS

This application claims priority under 35 USC §119(e) of U.S. provisional patent application 61/951,228, filed on Mar. 11, 2014, the specification of which is hereby incorporated by reference.

### BACKGROUND

(a) Field of the Invention

The subject matter disclosed generally relates to a carbon allotrope-silica composite material, processes for preparation thereof and method of uses thereof.

(b) Related Prior Art

Due to their unique physicochemical properties, carbon allotropes have emerged as novel materials apt to have a profound impact in many specialty applications. As an example, graphene, which is a one-atom-thick sheet of carbon atoms in a hexagonal arrangement, has a record thermal conductivity of about 5000 W·m⁻¹·K⁻¹ at room temperature (higher than diamond and carbon nanotubes), an extremely high specific area (theoretical value of 2630 m²·g⁻¹), a high intrinsic mobility (200,000 cm²·v⁻¹·s⁻¹), a unique Young's modulus (˜1.0 TPa) and a remarkable optical transmittance (97.7%). In this regard, carbon allotropes can be considered as templates of choice for the assembly of particles of interest on their surface. Indeed, the decoration of carbon allotropes with specific compounds and structures, such as silica nano- or microparticles, could increase their surface functionality and the tunability of their properties. The resulting materials can be used in numerous applications including electronics, electrochemistry, solar cells, biotechnology, etc. However, different studies reported to date on silica-carbon allotrope composite materials are mostly focused on dense silica particles, instead of hollow ones.

There is still a need for the design and use of hollow silica particles in the fabrication of such composite materials which can serve as a reservoir for different active agents including catalysts, polymer additives and other organic, inorganic or metallic compounds with specific properties.

### SUMMARY

The use of hollow silica particles in the fabrication of such composite materials is very interesting since the final product is much lighter and it can serve as a reservoir for different active agents including catalysts, polymer additives and other organic, inorganic or metallic compounds with specific properties. In terms of applications, a special focus has been paid in this invention on the use of silica microcapsules obtained from a previously reported process (International patent application publication No. WO2013/078551) or the above mentioned silica-carbon allotrope microparticles as advanced materials and their use in biotechnology as carriers for microorganisms and enzymes and for adsorption applications.

According to an embodiment, there is provided a carbon allotrope-silica composite material comprising:
a silica microcapsule comprising:
a silica shell having a thickness of from about 50 nm to about 500 μm, and a plurality of pores, the shell forming a capsule having a diameter from about 0.2 μm to about 1500 μm, and having a density of about 0.001 g/cm³ to about 1.0 g/cm³,
wherein the shell may comprise from about 0% to about 70% Q3 configuration, and from about 30% to about 100% Q4 configuration, or
wherein the shell may comprise from about 0% to about 60% T2 configuration and from about 40% to about 100% T3 configuration, or
wherein the shell may comprise a combination of T and Q configurations thereof, and
wherein an exterior surface of the capsule may be covered by a functional group;
and
a carbon allotrope attached to the silica microcapsule using a chemical process (in situ or post-functionalization in solution) or a physical process (plasma deposition).

According to another embodiment, there is provided a process for the preparation of a carbonallotrope-silica composite material comprising:
a) contacting an oxidized carbon allotrope with
a silica microcapsule, or
a silica precursor in a polar solvent in the presence of a catalyst for a sol-gel reaction
for a time sufficient and at a temperature sufficient obtain a formed carbon-allotrope silica composite material in a liquid phase.

According to another embodiment, there is provided a plasma deposition process for the preparation of a silica-carbon allotrope composite material comprising:
contacting silica microcapsules beforehand dispersed in an aqueous or an organic solution with
carbon allotrope precursors for a time, a pressure, a concentration and a power sufficient to obtain a formed silica-carbon allotrope composite material in the form of powder.

According to another embodiment, there is provided a carbon allotrope-silica composite material comprising:
a silica microcapsule comprising:
a silica shell having a thickness of from about 50 nm to about 500 μm, and a plurality of pores,
the shell forming a capsule having a diameter from about 0.2 μm to about 1500 μm, and having a density of about 0.001 g/cm³ to about 1.0 g/cm³, wherein the shell comprises from about 0% to about 70% Q3 configuration, and from about 30% to about 100% Q4 configuration, or wherein the shell comprises from about 0% to about 60% T2 configuration and from about 40% to about 100% T3 configuration, or
wherein the shell may comprise a combination of T and Q configurations thereof, and
wherein an exterior surface of the capsule may be covered by a functional group;
a carbon allotrope attached to the silica microcapsule.

According to another embodiment, there is provided a carbon allotrope-silica composite material comprising:
a carbon allotrope attached to a silica moiety comprising a silica nanoparticle having a diameter from about 5 nm to about 1000 nm, wherein an exterior surface of the silica nanoparticle may be covered by a functional group.

The thickness of the silica microcapsule may be from about 50 nm to about 240 μm.

The c diameter of the silica microcapsule may be from about 0.2 μm to about 500 μm.

The density of the silica microcapsule may be from about 0.01 g/cm³ to about 0.5 g/cm³.

The carbon allotrope may be attached covalently to the functional group of the silica particle.

The carbon allotrope may be attached non-covalently to the surface of the silica particle.

The functional group of the silica particle may be a hydroxyl group, a carboxylic acid group, a thiol group, an amino group, a benzylamino group, a chloropropyl group, a disulfide group, an epoxy group, a mercapto group, a methacrylate group, a vinyl group, and combinations thereof.

The carbon allotrope may be functionalized or not functionalized.

The functional group of the carbon allotrope may be a nitrogen-containing functional group, an oxygen containing functional group, a sulfur-containing functional group, a halogen-containing functional group and a combination thereof.

The nitrogen-containing functional group may be an amine group, a ketimine group, an aldimine group, an imide group, an azide group, an azo group, a cyanate group, an isocyanate group, a nitrate group, a nitrile group, a nitrite group, a nitroso group, a nitro group, a pyridyl group and a combination thereof.

The sulfur-containing functional group may be an sulfhydryl group, a sulfide group, a disulfide group, a sulfinyl group, a sulfonyl group, a sulfo group, a thiocyanate group, carbonothioyl group, carbonothioyl group and a combination thereof.

The oxygen-containing functional group may be an hydroxyl group, a carbonyl group, an aldehyde group, a carboxylate group, a carboxyl group, an ester group, a methoxy group, a peroxy group, an ether group, a carbonate ester and a combination thereof.

The halogen-containing functional group may be a fluoro, a chloro, a bromo, an iodo and a combination thereof.

The carbon allotrope may be chosen from graphite, graphene, a carbon nanofiber, a carbon nanotubes, a C60 fullerene, a C70 fullerene, a C76 fullerene, a C82 fullerene, a C84 fullerene, and a combination thereof.

The silica shell of the silica microcapsule may comprise from about 40% Q3 configuration and about 60% Q4 configuration, or from about 100% Q4 configuration.

The pores of the silica microcapsule have pore diameters from about 0.5 nm to about 100 nm.

The functional group of the silica microcapsule may be a hydroxyl group, an amino group, a benzylamino group, a chloropropyl group, a disulfide group, an epoxy group, a mercapto group, a methacrylate group, a vinyl group, and combinations thereof.

The functional group is provided by an organosilane chosen from a functional trimethoxysilane, a functional triethoxysilane, a functional tripropoxysilane, 3-aminopropyltriethoxysilane, vinyltriacetoxy silane, a vinyltrimethoxysilane, 3-glycidoxypropyltrimethoxysilane, 3-methacryloxypropyltrimethoxysilane, 3-chloropropyltriethoxysilane, a bis-(triethoxysilylpropyl)tetrasulfane, a methyltriethoxysilane, a n-octyltriethoxysilane, and a phenyltrimethoxysilane and combinations thereof.

The carbon allotrope-silica composite material may be loaded with a molecule.

The molecule may be a fluorescent molecule, a magnetic particle, a catalyst molecule, a biological macromolecule, or a combination thereof.

The magnetic molecule may be a magnetic nanoparticle.

According to another embodiment, there may be provided a process for the preparation of a carbon-allotrope silica composite material in solution comprising:
a) contacting an oxidized carbon allotrope with
   a silica microcapsule, or
   a silica precursor in a polar solvent in the presence of a catalyst for a sol-gel reaction
for a time sufficient and at a temperature sufficient obtain a formed carbon-allotrope silica composite material in a liquid phase.

The catalyst may be an acidic or alkali catalyst.

The polar solvent may be water, an alcohol, acetone, dimethylformamide (DMF), Dimethyl sulfoxide (DMSO) or a combination thereof.

The silica precursor may be an alkoxysilane.

The alkoxysilane may be methoxysilane, an ethoxysilane, a propoxysilane, an isopropoxysilane, an aryloxysilane, tetramethoxysilane (TMOS), tetraethoxysilane (TEOS), tetrapropoxysilane (TPOS) or a functional trimethoxy, triethoxysilane, tripropoxysilane including aminopropylsilane, aminoethylaminopropylsilane, vinyltrimethoxysilane, 3-chloropropyltriethoxysilane, 3-glycidoxypropyltrimethoxysilane, methacryloyloxypropyltrimethoxysilane, phenyltriethoxysilane, phenyltrimethoxysilane, glycidoxypropoxyltrimethoxysilane, glycidoxypropyltriethoxysilane, mercaptopropyltriethoxysilane, mercaptopropyltrimethoxysilane, aminopropyltrimethoxysilane, 3-aminopropyltriethoxysilane, 3-(2-aminoethylamino)propyltrimethoxysilane, 3-[2-(2-aminoethylamino)ethylamino]propyltrimethoxysilane, [2(cyclohexenyl)ethyl]triethoxysilane, vinyltrimethoxysilane, vinyltriethoxysilane or a mixture of any two or more of the above.

The acid catalyst may be chosen from HCl, acetic acid, and sulfuric acid, or a combination thereof.

The alkali catalyst may be chosen from sodium hydroxide, potassium hydroxide and ammonia, or a combination thereof.

The time sufficient may be from about 15 minutes to about 48 hours.

The temperature sufficient may be from about room temperature (24° C.) to about 100° C.

The oxidized carbon allotrope may be chosen from oxidized graphite, oxidized graphene, an oxidized carbon nanofiber, an oxidized carbon nanotubes, an oxidized C60 fullerene, an oxidized C70 fullerene, an oxidized C76 fullerene, an oxidized C82 fullerene, an oxidized C84 fullerene, and a combination thereof.

The process may further comprising step b) after step a)
b) washing the formed carbon-allotrope silica composite material to remove the acidic or alkali catalyst and an other impurity, to obtain washed carbon-allotrope silica composite material.

The process may further comprising step c) after step b):
c) separating the washed carbon-allotrope silica composite material from the liquid phase.

The process of may further comprising step d) after step c):
d) drying the washed carbon-allotrope silica composite material to obtain dried a carbon-allotrope silica composite material.

The silica microcapsule may comprise:
a silica shell having a thickness of from about 50 nm to about 500 μm, and a plurality of pores,
the shell forming a capsule having a diameter from about 0.2 μm to about 1500 μm, and having a density of about 0.001 g/cm³ to about 1.0 g/cm³, wherein the shell may comprise from about 0% to about 70% Q3 configuration, and from about 30% to about 100% Q4 configuration, or wherein the shell may comprise from about 0% to about 60% T2 configuration and from about 40% to about 100% T3 configuration, or wherein the shell may comprise a combination of T and Q configurations thereof, and
wherein an exterior surface of the capsule may be covered by a functional group;

The thickness of the silica microcapsule may be from about 50 nm to about 240 μm.

The diameter of the silica microcapsule may be from about 0.2 μm to about 500 μm.

The density of the silica microcapsule may be from about 0.01 g/cm³ to about 0.5 g/cm³.

The shell may comprise from about 40% Q3 configuration and about 60% Q4 configuration, or from about 100% Q4 configuration.

The pores may have pore diameters from about 0.5 nm to about 100 nm.

The functional group may be a hydroxyl group, an amino group, a benzylamino group, a chloropropyl group, a disulfide group, an epoxy group, a mercapto group, a methacrylate group, a vinyl group, and combinations thereof.

The functional group may be provided by an organosilane chosen from a functional trimethoxysilane, a functional triethoxysilane, a functional tripropoxysilane, 3-aminopropyltriethoxysilane, vinyltriacetoxy silane, a vinyltrimethoxysilane, 3-glycidoxypropyltrimethoxysilane, 3-methacryloxypropyltrimethoxysilane, 3-chloropropyltriethoxysilane, a bis-(triethoxysilylpropyl)tetrasulfane, a methyltriethoxysilane, a n-octyltriethoxysilane, and a phenyltrimethoxysilane and combinations thereof.

According to another embodiment, there is provided a process for the preparation of a carbon-allotrope silica composite material using a plasma deposition process, comprising:
a) contacting a silica microcapsule with a plasmagenic gas comprising a carbon precursor, or a carbon precursor in the presence of a nitrogen precursor, an oxygen precursor, or a sulfur precursor, or a combination thereof,
   for a time sufficient, at a power sufficient, a concentration, and a pressure sufficient to deposit a carbon allotrope onto the surface of the silica microcapsule to form the carbon-allotrope silica composite material.

The carbon precursor may be chosen from a cyclic hydrocarbon, an aliphatic hydrocarbon, a branched hydrocarbon, a halogenated hydrocarbon, and mixtures thereof.

The the aliphatic hydrocarbon may be methane.

The carbon precursor may be injected at a pressure of about 172.37 kPa to about 517.11 kPa.

The flow rate of the plasmagenic gas may be from about 0.1 slpm to about 1.5 slpm.

The flow rate of the plasmagenic gas may be from about 0.4 slpm to about 0.9 slpm.

The process may be further comprising injecting in the plasmagenic gas a sulfur-containing precursor, a nitrogen-containing precursor, an oxygen-containing precursor, a halogen-containing precursor, or a combination thereof.

The sulfur-containing precursor may be chosen from a sulfate, a persulfate, a sulfide, a sulfite, a sulfur oxide, a organosulfur compound, a thionyl compound, a thiosulfates, a thiocyanate, a isothiocyanate, a sulfuryl compound, a sulfonium compound, or a combination thereof.

The nitrogen-containing precursor may be chosen from nitrogen (gas N₂), ammonia, an amine, an amide, an imine, an ammonium compound, an azide, a cyanate, a cyanide, a hydrazine, a nitrate, a nitrite, a nitride, a nitrosyl compound, an isocyanate, a nitrogen halide, an organonitrogen compound, a thiocyanate, a thioureas, or a combination thereof.

The oxygen-containing precursor may be chosen from oxygen (gas O₂), a oxide, a peroxide, an alcohol, an ether, a ketone, an aldehyde, a carboxylic acid, an ether, an acid anhydride, an amides, or a combination thereof.

The halogen-containing precursor may be chosen from a bromide compound, a chlorine compound, a fluorine compound, an iodine compound, an halide, an interhalogen compound, or a combination thereof.

The process may comprise a sheath gas and the sheath gas may be chosen from He, Ne, Ar, Xe, N₂, and a combination thereof.

The sheath gas may be Ar.

The sheath gas may be injected at a pressure of from about 172.37 kPa to about 517.11 kPa.

The sheath gas may be injected at a pressure of from about 275.79 kPa to about 413.69 kPa.

The carrier gas may comprise from about 1.7% to about 8% v/v carbon precursor vapor.

The carrier gas may comprise from about 4% to about 8% v/v carbon precursor vapor.

The power sufficient may be from about 1 to about 50 kW.

The power sufficient may be from about 5 to about 20 kW.

The pressure sufficient may be from about 13.33 kPa to about 61.33 kPa.

The time sufficient may be from about 1 to about 60 minutes.

According to another embodiment, there is provided a material comprising:
a carbon allotrope-silica composite material according to the present invention,
a silica microcapsule comprising:
   a silica shell having a thickness of from about 50 nm to about 500 μm, and a plurality of pores,
   the shell forming a capsule having a diameter from about 0.2 μm to about 1500 μm, and having a density of about 0.001 g/cm³ to about 1.0 g/cm³,
   wherein the shell may comprise from about 0% to about 70% Q3 configuration, and from about 30% to about 100% Q4 configuration, or
   wherein the shell may comprise from about 0% to about 60% T2 configuration and from about 40% to about 100% T3 configuration, or
   wherein the shell may comprise a combination of T and Q configurations thereof, and
   wherein an exterior surface of the capsule may be covered by a functional group,
or a combination thereof, and
   a cell, an enzyme, a viral particle, or a combination thereof.

The material may be for carrying a cell, an enzyme, a viral particle or a combination thereof.

The cell may be a prokaryotic cell or a eukaryotic cell.

The prokaryotic cell may be chosen from a bacterial cell, and an archaea cell.

The eukaryotic cell may be chosen from a fungal cell, a protozoan cell, an insect cell, a plant cell, and a mammalian cell.

The shell may comprise from about 40% Q3 configuration and about 60% Q4 configuration, or from about 100% Q4 configuration.

The pores of the silica microcapsule have pore diameters from about 0.5 nm to about 100 nm.

The functional group may be a hydroxyl group, an amino group, a benzylamino group, a chloropropyl group, a disulfide group, an epoxy group, a mercapto group, a methacrylate group, a vinyl group, and combinations thereof

The functional group may be provided by an organosilane chosen from a functional trimethoxysilane, a functional triethoxysilane, a functional tripropoxysilane, 3-aminopropyltriethoxysilane, vinyltriacetoxy silane, a vinyltrimethoxysilane, 3-glycidoxypropyltrimethoxysilane, 3-methacryloxypropyltrimethoxysilane, 3-chloropropyltriethoxysilane, a bis-(triethoxysilylpropyl)tetrasulfane, a methyltriethoxysilane, a n-octyltriethoxysilane, and a phenyltrimethoxysilane and combinations thereof.

According to another embodiment, there is provided a process for the preparation of a material comprising:
a) contacting
a carbon allotrope-silica composite material of the present invention, or
a silica microcapsule comprising:
   a silica shell having a thickness of from about 50 nm to about 500 μm, and a plurality of pores,
   the shell forming a capsule having a diameter from about 0.2 μm to about 1500 μm, and having a density of about 0.001 g/cm³ to about 1.0 g/cm³,
   wherein the shell may comprise from about 0% to about 70% Q3 configuration, and from about 30% to about 100% Q4 configuration, or
   wherein the shell may comprise from about 0% to about 60% T2 configuration and from about 40% to about 100% T3 configuration, or
   wherein the shell may comprise a combination of T and Q configurations thereof, and
   wherein an exterior surface of the capsule may be covered by a functional group,
or a combination thereof,
   with a cell, an enzyme, or a viral particle, and incubating for a time sufficient for binding of the microorganism, enzyme, or viral particle to the carbon allotrope-silica composite material, the silica microcapsule or the combination thereof.

The shell may comprise from about 40% Q3 configuration and about 60% Q4 configuration, or from about 100% Q4 configuration.

The pores of the silica microcapsule have pore diameters from about 0.5 nm to about 100 nm.

The functional group may be a hydroxyl group, an amino group, a benzylamino group, a chloropropyl group, a disulfide group, an epoxy group, a mercapto group, a methacrylate group, a vinyl group, and combinations thereof

The functional group may be provided by an organosilane chosen from a functional trimethoxysilane, a functional triethoxysilane, a functional tripropoxysilane, 3-aminopropyltriethoxysilane, vinyltriacetoxy silane, a vinyltrimethoxysilane, 3-glycidoxypropyltrimethoxysilane, 3-methacryloxypropyltrimethoxysilane, 3-chloropropyltriethoxysilane, a bis-(triethoxysilylpropyl)tetrasulfane, a methyltriethoxysilane, a n-octyltriethoxysilane, and a phenyltrimethoxysilane and combinations thereof.

The cell may be chosen from a prokaryotic cell or a eukaryotic cell.

The prokaryotic cell may be chosen from a bacterial cell, and an archaea cell.

The eukaryotic cell may be chosen from a fungal cell, a protozoan cell, an insect cell, a plant cell, and a mammalian cell.

The bacterial cell may be chosen from the following phyla: an Acidobacteria, an Actinobacteria, an Aquificae, an Bacteroidetes, an Caldiserica, an Chlamydiae, an Chlorobi, an Chloroflexi, an Chrysiogenetes, an Cyanobacteria, an Deferribacteres, an Deinococcus-Thermus, an Dictyoglomi, an Elusimicrobia, an Fibrobacteres, an Firmicutes, an Fusobacteria, an Gemmatimonadetes, an Lentisphaerae, an Nitrospira, an Planctomycetes, an Proteobacteria, an Spirochaetes, an Synergistetes, an Tenericutes, an Thermodesulfobacteria, an Thermotogae, an Verrucomicrobia, or a combination thereof.

The bacterial cell may be chosen from the following genera: Pseudomonas, Rhodopseudomonas, Acinetobacter, Mycobacterium, Corynebacterium, Arthrobacterium, Bacillius, Flavorbacterium, Nocardia, Achromobacterium, Alcaligenes, Vibrio, Azotobacter, Beijerinckia, Xanthomonas. Nitrosomonas, Nitrobacter, Methylosinus, Methylococcus, Actinomycetes and Methylobacter.

The archaeal cell may be chosen from the following phyla: an Euryarchaeota, an Crenarchaeota, an Korarchaeota, an Nanoarchaeota, or a combination thereof.

The fungal cell may be chosen from phyla including a Blastocladiomycota, a Chytridiomycota, a Glomeromycota, a Microsporidia, a Neocallimastigomycota, an Ascomycota, a Basidiomycota, or a combination thereof.

The fungal cell may be chosen from the following genera: Saccaromyces, Pichia, Brettanomyces, Yarrowia, Candida, Schizosaccharomyces, Torulaspora, Zygosaccharomyces Aspergillus, Rhizopus, Trichoderma, Monascus, Penicillium, Fusarium, Geotrichum, Neurospora, Rhizomucor, and Tolupocladium.

The protozoan cell may be chosen from the following phyla: Percolozoa, Euglenozoa, Ciliophora, Mioza, Dinoza, Apicomplexa, Opalozoa, Mycetozoa, Radiozoa, Heliozoa, Rhizopoda, Neosarcodina, Reticulosa, Choanozoa, Myxosporida, Haplosporida, Paramyxia.

The eukaryotic cell may be from an algae.

The enzyme may be chosen from a oxidoreductase, a transferase, a hydrolase, a lyase, an isomerase, a ligase, a polymerase or a combination thereof.

The process may be carried in a biological reactor.

The biological reactor may be chosen from a fermentation batch reactor, an enzymatic batch reactor, a nitrification reactor, a digester reactor, a membrane bioreactor (MBR), a moving bed bioreactor (MBBR), a fluid bed reactor (FBR), a continuous stirred reactor (CSTR), a plug flow reactor (PFR) and a sequential batch reactor (SBR).

The method may be an anaerobic or an aerobic method.

According to another embodiment, there is provided a material obtained from the processes of the present invention.

According to another embodiment, there is provided a method of cell growth comprising incubating a material according to the present invention, in a sterile growth medium to obtain the cell.

According to another embodiment, there is provided a method for performing an enzymatic reaction comprising incubating a material according to the present invention, in a reaction medium.

According to another embodiment, there is provided a method for performing a fermentation reaction comprising incubating a material according to the present invention, in a fermentation reaction medium to obtain a fermentation product.

The growth may be a sporulation reaction to obtain spores.

According to another embodiment, there is provided a method for decontamination of a contaminated fluid comprising incubating a material according to the present invention, in the contaminated fluid.

The method may be carried in a biological reactor.

The biological reactor may be chosen from a fermentation batch reactor, an enzymatic batch reactor, a nitrification reactor, a digester reactor, a membrane bioreactor (MBR), a moving bed bioreactor (MBBR), a fluid bed reactor (FBR), a continuous stirred reactor (CSTR), a plug flow reactor (PFR) and a sequential batch reactor (SBR).

According to another embodiment, there is provided a process for the preparation of a material comprising:
a) contacting
a carbon allotrope-silica composite material of the present invention or,
a silica microcapsule comprising:
   a silica shell having a thickness of from about 50 nm to about 500 μm, and a plurality of pores,
   the shell forming a capsule having a diameter from about 0.2 μm to about 1500 μm, and having a density of about 0.001 g/cm³ to about 1.0 g/cm³,
   wherein the shell may comprise from about 0% to about 70% Q3 configuration, and from about 30% to about 100% Q4 configuration, or
   wherein the shell may comprise from about 0% to about 60% T2 configuration and from about 40% to about 100% T3 configuration, or
   wherein the shell may comprise a combination of T and Q configurations thereof, and
   wherein an exterior surface of the capsule may be covered by a functional group,
or a combination thereof,
   with a molecule for adsorption of the molecule to the carbon allotrope-silica composite material, the silica microcapsule or the combination thereof.

The thickness of the silica microcapsule may be from about 50 nm to about 240 μm.

The diameter of the silica microcapsule may be from about 0.2 μm to about 500 μm.

The density of the silica microcapsule may be from about 0.01 g/cm³ to about 0.5 g/cm³.

The shell may comprise from about 40% Q3 configuration and about 60% Q4 configuration, or from about 100% Q4 configuration.

The pores of the silica microcapsule have pore diameters from about 0.5 nm to about 100 nm.

The functional group may be a hydroxyl group, an amino group, a benzylamino group, a chloropropyl group, a disulfide group, an epoxy group, a mercapto group, a methacrylate group, a vinyl group, and combinations thereof

The functional group may be provided by an organosilane chosen from a functional trimethoxysilane, a functional triethoxysilane, a functional tripropoxysilane, 3-aminopropyltriethoxysilane, vinyltriacetoxy silane, a vinyltrimethoxysilane, 3-glycidoxypropyltrimethoxysilane, 3-methacryloxypropyltrimethoxysilane, 3-chloropropyltriethoxysilane, a bis-(triethoxysilylpropyl)tetrasulfane, a methyltriethoxysilane, a n-octyltriethoxysilane, and a phenyltrimethoxysilane and combinations thereof.

The molecule may be a fluorescent molecule, a magnetic particle, a catalyst molecule, a biological macromolecule, or a combination thereof.

The following terms are defined below.

### Definitions

“Alkyl”, as well as other groups having the prefix “alk”, such as alkoxy and alkanoyl, means carbon chains which may be linear or branched, and combinations thereof, unless the carbon chain is defined otherwise. Examples of alkyl groups include methyl, ethyl, propyl, isopropyl, butyl, sec- and tert-butyl, pentyl, hexyl, heptyl, octyl, nonyl, and the like. Where the specified number of carbon atoms permits, e.g., from C₃₋₁₀, the term alkyl also includes cycloalkyl groups, and combinations of linear or branched alkyl chains combined with cycloalkyl structures. When no number of carbon atoms is specified, C₁₋₆ is intended.

“Cycloalkyl” is a subset of alkyl and means a saturated carbocyclic ring having a specified number of carbon atoms. Examples of cycloalkyl include cyclopropyl, cyclobutyl, cyclopentyl, cyclohexyl, cycloheptyl, cyclooctyl, and the like. A cycloalkyl group generally is monocyclic unless stated otherwise. Cycloalkyl groups are saturated unless otherwise defined.

The term “alkoxy” refers to straight or branched chain alkoxides of the number of carbon atoms specified (e.g., C₁₋₆ alkoxy), or any number within this range [i.e., methoxy (MeO—), ethoxy, isopropoxy, etc.].

The term “alkylthio” refers to straight or branched chain alkylsulfides of the number of carbon atoms specified (e.g., C₁₋₆ alkylthio), or any number within this range [i.e., methylthio (MeS—), ethylthio, isopropylthio, etc.].

The term “alkylamino” refers to straight or branched alkylamines of the number of carbon atoms specified (e.g., C₁₋₆ alkylamino), or any number within this range [i.e., methylamino, ethylamino, isopropylamino, t-butylamino, etc.].

The term “alkylsulfonyl” refers to straight or branched chain alkylsulfones of the number of carbon atoms specified (e.g., C₁₋₆ alkylsulfonyl), or any number within this range [i.e., methylsulfonyl (MeSO₂⁻), ethylsulfonyl, isopropylsulfonyl, etc.].

The term “alkylsulfinyl” refers to straight or branched chain alkylsulfoxides of the number of carbon atoms specified (e.g., C₁₋₆ alkylsulfinyl), or any number within this range [i.e., methylsulfinyl (MeSO—), ethylsulfinyl, isopropylsulfinyl, etc.].

The term “alkyloxycarbonyl” refers to straight or branched chain esters of a carboxylic acid derivative of the present invention of the number of carbon atoms specified (e.g., C₁₋₆ alkyloxycarbonyl), or any number within this range [i.e., methyloxycarbonyl (MeOCO⁻), ethyloxycarbonyl, or butyloxycarbonyl].

“Aryl” means a mono- or polycyclic aromatic ring system containing carbon ring atoms. The preferred aryls are monocyclic or bicyclic 6-10 membered aromatic ring systems. Phenyl and naphthyl are preferred aryls. The most preferred aryl is phenyl.

“Heterocyclyl” refer to saturated or unsaturated non-aromatic rings or ring systems containing at least one heteroatom selected from O, S and N, further including the oxidized forms of sulfur, namely SO and SO₂. Examples of heterocycles include tetrahydrofuran (THF), dihydrofuran, 1,4-dioxane, morpholine, 1,4-dithiane, piperazine, piperidine, 1,3-dioxolane, imidazolidine, imidazoline, pyrroline, pyrrolidine, tetrahydropyran, dihydropyran, oxathiolane, dithiolane, 1,3-dioxane, 1,3-dithiane, oxathiane, thiomorpholine, 2-oxopiperidin-1-yl, 2-oxopyrrolidin-1-yl, 2-oxoazetidin-1-yl, 1,2,4-oxadiazin-5(6H)-one-3-yl, and the like.

“Heteroaryl” means an aromatic or partially aromatic heterocycle that contains at least one ring heteroatom selected from O, S and N. Heteroaryls thus include heteroaryls fused to other kinds of rings, such as aryls, cycloalkyls and heterocycles that are not aromatic. Examples of heteroaryl groups include: pyrrolyl, isoxazolyl, isothiazolyl, pyrazolyl, pyridyl, oxazolyl, oxadiazolyl (in particular, 1,3,4-oxadiazol-2-yl and 1,2,4-oxadiazol-3-yl), thiadiazolyl, thiazolyl, imidazolyl, triazolyl, tetrazolyl, furyl, triazinyl, thienyl, pyrimidyl, benzisoxazolyl, benzoxazolyl, benzothiazolyl, benzothiadiazolyl, dihydrobenzofuranyl, indolinyl, pyridazinyl, indazolyl, isoindolyl, dihydrobenzothienyl, indolizinyl, cinnolinyl, phthalazinyl, quinazolinyl, naphthyridinyl, carbazolyl, benzodioxolyl, quinoxalinyl, purinyl, furazanyl, isobenzylfuranyl, benzimidazolyl, benzofuranyl, benzothienyl, quinolyl, indolyl, isoquinolyl, dibenzofuranyl, and the like. For heterocyclyl and heteroaryl groups, rings and ring systems containing from 3-15 atoms are included, forming 1-3 rings.

“Halogen” refers to fluorine, chlorine, bromine and iodine. Chlorine and fluorine are generally preferred. Fluorine is most preferred when the halogens are substituted on an alkyl or alkoxy group (e.g. CF₃O and CF₃CH₂O).

The term <<composition>> as used herein is intended to encompass a product comprising the specified ingredients in the specified amounts, as well as any product which results, directly or indirectly, from combination of the specified ingredients in the specified amounts. Such term in relation to pharmaceutical composition is intended to encompass a product comprising the active ingredient(s) and the inert ingredient(s) that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing a compound of the present invention and a pharmaceutically acceptable carrier. By “pharmaceutically acceptable” or “acceptable” it is meant the carrier, diluent or excipient must be compatible with the other ingredients of the formulation and not deleterious to the recipient thereof.

The term “growth medium” is intended to mean is a liquid or gel designed to support the growth of microorganisms or cells. There are two major types of growth media: those used for cell culture, which use specific cell types derived from eukaryotic multicellular organism such as plants, insects or animals, and microbiological culture, which are used for growing microorganisms, such as bacteria fungi or algae. The most common growth media for microorganisms are nutrient broths and agar plates; specialized media are sometimes required for microorganism and cell culture growth. Some organisms, termed fastidious organisms, require specialized environments due to complex nutritional requirements. Viruses, for example, are obligate intracellular parasites and require a growth medium containing living cells. Thus, the term “growth medium” is intended to include any and all nutrients or compounds that are necessary for the growth or maintenance of microorganisms, cells or viruses therein.

The term “reaction medium” or “reaction solution” is intended to mean a medium or solution which contains all the necessary ingredients for a chemical reaction to occur. For example, the medium or solution may contain salts or minerals, chemicals to maintain a specific pH (e.g. buffering reagents), chemical factors and cofactors, etc., all of which may be dissolved in a solvent such as water or any other suitable solvent. According to an embodiment, the reaction may be an enzymatic reaction.

The term “fermentation medium” is intended to mean a medium or solution in which fermentation may readily occur in the presence of the appropriate microorganisms. Similar to the “growth” medium above, the fermentation medium may contain all the necessary ingredients (nutrients) necessary to support the survival of microorganisms or cells therein.

The term “virus particle”, also known as “virion” or “virus” is intended to mean particles composed of two or three parts: i) the genetic material made from either DNA or RNA, long molecules that carry genetic information; ii) a protein coat that protects these genes; and in some cases iii) an envelope of lipids that surrounds the protein coat when they are outside a cell. The shapes of viruses range from simple helical and icosahedral forms to more complex structures. The average virus is about one one-hundredth the size of the average bacterium. Most viruses are too small to be seen directly with an optical microscope.

The term “cell” is intended to mean the basic structural, functional, and biological unit of all known living organisms. Cells are the smallest unit of life that can replicate independently, and are often called the “building blocks of life”. According to the present inventions, the cells may be any cells from prokaryotic or eukaryotic origins, such as bacterial cells or archeal cells, as well as insect, plant, fungal, mammalian, or any other cells.

Before describing the present invention in detail, a number of terms will be defined. As used herein, the singular forms “a”, “an”, and “the” include plural referents unless the context clearly dictates otherwise.

It is noted that terms like “preferably”, “commonly”, and “typically” are not utilized herein to limit the scope of the claimed invention or to imply that certain features are critical, essential, or even important to the structure or function of the claimed invention. Rather, these terms are merely intended to highlight alternative or additional features that can or cannot be utilized in a particular embodiment of the present invention.

For the purposes of describing and defining the present invention it is noted that the term “substantially” is utilized herein to represent the inherent degree of uncertainty that can be attributed to any quantitative comparison, value, measurement, or other representation. The term “substantially” is also utilized herein to represent the degree by which a quantitative representation can vary from a stated reference without resulting in a change in the basic function of the subject matter at issue.

Features and advantages of the subject matter hereof will become more apparent in light of the following detailed description of selected embodiments, as illustrated in the accompanying figures. As will be realized, the subject matter disclosed and claimed is capable of modifications in various respects, all without departing from the scope of the claims. Accordingly, the drawings and the description are to be regarded as illustrative in nature, and not as restrictive, the full scope of the subject matter being set forth in the claims.

### BRIEF DESCRIPTION OF THE DRAWINGS

Further features and advantages of the present disclosure will become apparent from the following detailed description, taken in combination with the appended drawings, in which:

FIG. 1 shows SEM image and the corresponding EDS spectra of graphene flakes covered with silica nanoparticles;

FIG. 2 shows TEM images of graphene sheets produced using plasma deposition process, according to embodiments of the present invention (Table 1);

FIG. 3 shows SEM images of a) a silica microcapsule and b) a silica-graphene microparticle produced using plasma deposition process, according to embodiments of the present invention (Table 2);

FIG. 4 shows SEM images of silica-graphene composite materials functionalized with nitrogen-containing functional groups via plasma deposition process using a) NH₃ and b) N₂ as nitrogen precursors;

FIG. 5 shows XPS spectra of silica-graphene composite materials functionalized with nitrogen-containing functional groups via plasma deposition process using NH₃ and N₂ as nitrogen precursors;

FIG. 6 shows XPS high resolution spectra of the N 1s peak from samples from a) NH₃ and b) N₂ as nitrogen precursors;

FIG. 7 shows optical micrographs of bacteria a) without a carrier and b) with silica microcapsules at 400× magnification;

FIG. 8 shows optical micrographs of bacteria in the presence of silica microcapsules prewashed with a LB medium at a) 1000× and b) 100× magnification;

FIG. 9 shows the bio-production of methane in using bacteria with silica microcapsules and chitosan as carriers;

FIG. 10 shows the enzymatic activity of protease obtain from a fermentation in the presence of silica microcapsules;

FIG. 11 shows yeast fermentation with silica microcapsules: a) after 48 hours of incubation, samples 1 to 6 from left to right; b) after 30 minutes of sedimentation, samples 1 to 6 from left to right and c) after saline washing by inversion, sample 2 to 6 from left to right;

FIG. 12 shows optical microscopy micrographs of Bacillus subtilis incubated for 24 hours with silica-carbon allotrope composite microparticles at a) 100× and b) 1000× magnification;

FIG. 13 shows the ammonia consumption using a nitrifying consortium of bacteria with and without silica microcapsules;

FIG. 14 shows Scheme 1 which is a schematic drawing of the plasma torch equipment;

FIG. 15 shows Scheme 2 which is a schematic drawings of different configurations used for the deposition of graphene onto silica microcapsules.

It will be noted that throughout the appended drawings, like features are identified by like reference numerals.

### DETAILED DESCRIPTION

This invention comprises two parts described as follow. In the first part, different carbon allotrope-silica composite materials are provided. The above mentioned carbon allotropes can be chosen from graphite, graphene, carbon nanofibers, carbon nanotubes, C60 fullerene, C70 fullerene, etc. For the preparation of these composite materials, different approaches based on chemical or physical processes have been considered. These approaches include:

Chemical grafting of silica microcapsules obtained from International patent Application publication No. WO2013/078551 with allotropes of carbon.

In situ synthesis of silica nanoparticles onto the surface of carbon allotropes via the sol-gel process.

Formation and in situ coating of carbon allotropes onto silica microcapsules using plasma deposition.

Formation and in situ coating of functionalized carbon allotropes onto silica microcapsules using plasma deposition.

The second part of this invention describes the use of silica microcapsules obtained as described in International patent Application publication No. WO2013/078551 or the above obtained silica-carbon allotrope composites as advanced materials (e.g. electrical and/or thermal conductive fillers for silica-carbon allotrope microparticles) and their use in bio-processes (e.g. as carriers for any type of cells, including microorganisms, and eukaryotic cell derived from multicellular organisms, enzymes, and/or viral particles) or for adsorption of specific molecules.

### Preparation of Silica-Carbon Allotrope Composite Materials

The present invention provides various silica-carbon allotrope composite materials intended to be used in numerous specialty applications. To this end, different chemical or physical approaches giving rise to various morphologies have been considered.

### Chemical Processes

According to an embodiment, a first approach involves a chemical grafting of silica microcapsules with carbon allotropes including graphite, graphene, carbon nanofibers, carbon nanotubes, C60, C70, C76, C82 and C84 fullerenes, etc, and their combination. The initial silica microcapsules, produced as described in International patent Application publication No. WO2013/078551, are hollow and their size can range from 0.2 to 1500 microns depending on the intended application. These silica microcapsules intrinsically contain hydroxyl groups on their surface, which allow further surface modification (attachment of functional groups including amino, vinyl, epoxy, disulfide, etc.) using functional organosilanes. The presence of these functional groups on the surface of silica particles is primordial for a covalent tethering of carbon allotropes. Before being attached with silica microparticles, carbon allotropes have to be oxidized under strong oxidizing conditions (HNO₃, KClO₃, KMO₄/H₂SO₄, H₂CrO₄/H₂SO₄, etc.), as described by the well-known Hummers method (Hummers, W. and Offeman, R.; J. Am. Chem. Soc. 1958, 80, 1339). This results in the formation of various oxide-containing species including hydroxyl, carboxyl and epoxy groups. As a result, the resulting functional groups can covalently react with those present on the surface of silica particles in order to obtain covalently linked silica-carbon allotrope composite materials. As an example, taking advantage of carboxylic acids present on the surface of oxidized carbon allotropes, various coupling reactions can be considered. These coupling reactions require activation of the carboxylic acid group using thionyl chloride (SOCl₂), 1-ethyl-3-(3-dimethylaminopropyl)-carbodiimide (EDC), N,N′ dicyclohexylcarbodiimide (DCC), 2-(7-aza-1H-benzotriazole-1-yl)-1,1,3,3-tetramethyluronium hexafluorophosphate (HATU), etc. A subsequent reaction with nucleophilic species such as amine or hydroxyl groups available on the silica surface produces covalent bonding via the formation of amides or esters. In addition to carboxylic acids, epoxy groups present on the surface of oxidized carbon allotropes can be easily modified through ring-opening reactions under various conditions, using amine-functionalized silica microcapsules.

The microcapsules which may be used in the present invention have an average diameter from about 0.2μm to about 1500 μm. The diameter of the microcapsule may be from about 0.2 μm to about 1500 μm, or from about 0.2 μm to about 1000 μm, or from about 0.2 μm to about 1500 μm, or from about 0.2 μm to about 900 μm, or from about 0.2 μm to about 800 μm, or from about 0.2 μm to about 700 μm, or from about 0.2 μm to about 600 μm, or from about 0.2 μm to about 500 μm, or from about 0.2 μm to about 400 μm, or from about 0.2 μm to about 300 μm, or from about 0.2 μm to about 200 μm, or from about 0.2 μm to about 100 μm, or from about 0.2 μm to about 90 μm, or from about 0.2 μm to about 80 μm, or from about 0.2 μm to about 70 μm, or from about 0.2 μm to about 60 μm, or from about 0.2 μm to about 50 μm, or from about 0.2 μm to about 40 μm, or from about 0.2 μm to about 30 μm, or from about 0.2 μm to about 20 μm, or from about 0.2 μm to about 15 μm, or from about 0.2 μm to about 10 μm, or from about 0.2 μm to about 5 μm, or from about 0.2 μm to about 2 μm, 0.5 μm to about 1500 μm, or from about 0.5μm to about 1000 μm, or from about 0.5μm to about 1500 μm, or from about 0.5μm to about 900 μm, or from about 0.5μm to about 800 μm, or from about 0.5μm to about 700 μm, or from about 0.5μm to about 600 μm, or from about 0.5μm to about 500 μm, or from about 0.5μm to about 400 μm, or from about 0.5μm to about 300 μm, or from about 0.5μm to about 200 μm, or from about 0.5μm to about 100 μm, or from about 0.5μm to about 90 μm, or from about 0.5μm to about 80 μm, or from about 0.5μm to about 70 μm, or from about 0.5μm to about 60 μm, or from about 0.5μm to about 50 μm, or from about 0.5μm to about 40 μm, or from about 0.5μm to about 30 μm, or from about 0.5μm to about 20 μm, or from about 0.5μm to about 15 μm, or from about 0.5μm to about 10 μm, or from about 0.5μm to about 5 μm, or from about 0.5μm to about 2 μm, 1 μm to about 1500 μm, or from about 1 μm to about 1000 μm, or from about 1μm to about 1500 μm, or from about 1μm to about 900 μm, or from about 1μm to about 800 μm, or from about 1μm to about 700 μm, or from about 1μm to about 600 μm, or from about 1μm to about 500 μm, or from about 1μm to about 400 μm, or from about 1μm to about 300 μm, or from about 1μm to about 200 μm, or from about 1μm to about 100 μm, or from about 1μm to about 90 μm, or from about 1μm to about 80 μm, or from about 1μm to about 70 μm, or from about 1μm to about 60 μm, or from about 1μm to about 50 μm, or from about 1μm to about 40 μm, or from about 1 μm to about 30 μm, or from about 1μm to about 20 μm, or from about 1μm to about 15 μm, or from about 1μm to about 10 μm, or from about 1 μm to about 5 μm, or from about 1μm to about 2 μm, 2 μm to about 1500 μm, or from about 2 μm to about 1000 μm, or from about 2 μm to about 1500 μm, or from about 2 μm to about 900 μm, or from about 2 μm to about 800 μm, or from about 2 μm to about 700 μm, or from about 2 μm to about 600 μm, or from about 2 μm to about 500 μm, or from about 2 μm to about 400 μm, or from about 2 μm to about 300 μm, or from about 2 μm to about 200 μm, or from about 2 μm to about 100 μm, or from about 2 μm to about 90 μm, or from about 2 μm to about 80 μm, or from about 2 μm to about 70 μm, or from about 2 μm to about 60 μm, or from about 2 μm to about 50 μm, or from about 2 μm to about 40 μm, or from about 2 μm to about 30 μm, or from about 2 μm to about 20 μm, or from about 2 μm to about 15 μm, or from about 2 μm to about 10 μm, or from about 2 μm to about 5 μm, 3 μm to about 1500 μm, or from about 3 μm to about 1000 μm, or from about 3 μm to about 1500 μm, or from about 3 μm to about 900 μm, or from about 3 μm to about 800 μm, or from about 3 μm to about 700 μm, or from about 3 μm to about 600 μm, or from about 3 μm to about 500 μm, or from about 3 μm to about 400 μm, or from about 3 μm to about 300 μm, or from about 3 μm to about 200 μm, or from about 3 μm to about 100 μm, or from about 3 μm to about 90 μm, or from about 3 μm to about 80 μm, or from about 3 μm to about 70 μm, or from about 3 μm to about 60 μm, or from about 3 μm to about 50 μm, or from about 3 μm to about 40 μm, or from about 3 μm to about 30 μm, or from about 3 μm to about 20 μm, or from about 3 μm to about 15 μm, or from about 3 μm to about 10 μm, or from about 3 μm to about 5 μm, 4 μm to about 1500 μm, or from about 4 μm to about 1000 μm, or from about 4 μm to about 1500 μm, or from about 4 μm to about 900 μm, or from about 4 μm to about 800 μm, or from about 4 μm to about 700 μm, or from about 4 μm to about 600 μm, or from about 4 μm to about 500 μm, or from about 4 μm to about 400 μm, or from about 4 μm to about 300 μm, or from about 4 μm to about 200 μm, or from about 4 μm to about 100 μm, or from about 4 μm to about 90 μm, or from about 4 μm to about 80 μm, or from about 4 μm to about 70 μm, or from about 4 μm to about 60 μm, or from about 4 μm to about 50 μm, or from about 4 μm to about 40 μm, or from about 4 μm to about 30 μm, or from about 4 μm to about 20 μm, or from about 4 μm to about 15 μm, or from about 4 μm to about 10 μm, or from about 4 μm to about 5 μm, 5 μm to about 1500 μm, or from about 5 μm to about 1000 μm, or from about 5 μm to about 1500 μm, or from about 5 μm to about 900 μm, or from about 5 μm to about 800 μm, or from about 5 μm to about 700 μm, or from about 5 μm to about 600 μm, or from about 5 μm to about 500 μm, or from about 5 μm to about 400 μm, or from about 5 μm to about 300 μm, or from about 5 μm to about 200 μm, or from about 5 μm to about 100 μm, or from about 5 μm to about 90 μm, or from about 5 μm to about 80 μm, or from about 5 μm to about 70 μm, or from about 5 μm to about 60 μm, or from about 5 μm to about 50 μm, or from about 5 μm to about 40 μm, or from about 5 μm to about 30 μm, or from about 5 μm to about 20 μm, or from about 5 μm to about 15 μm, or from about 5 μm to about 10 μm, 10 μm to about 1500 μm, or from about 10 μm to about 1000 μm, or from about 10 μm to about 1500 μm, or from about 10 μm to about 900 μm, or from about 10 μm to about 800 μm, or from about 10 μm to about 700 μm, or from about 10 μm to about 600 μm, or from about 10 μm to about 500 μm, or from about 10 μm to about 400 μm, or from about 10 μm to about 300 μm, or from about 10 μm to about 200 μm, or from about 10 μm to about 100 μm, or from about 10 μm to about 90 μm, or from about 10 μm to about 80 μm, or from about 10 μm to about 70 μm, or from about 10 μm to about 60 μm, or from about 10 μm to about 50 μm, or from about 10 μm to about 40 μm, or from about 10 μm to about 30 μm, or from about 10 μm to about 20 μm, or from about 10 μm to about 15 μm, 15 μm to about 1500 μm, or from about 15 μm to about 1000 μm, or from about 15 μm to about 1500 μm, or from about 15 μm to about 900 μm, or from about 15 μm to about 800 μm, or from about 15 μm to about 700 μm, or from about 15 μm to about 600 μm, or from about 15 μm to about 500 μm, or from about 15 μm to about 400 μm, or from about 15 μm to about 300 μm, or from about 15 μm to about 200 μm, or from about 15 μm to about 100 μm, or from about 15 μm to about 90 μm, or from about 15 μm to about 80 μm, or from about 15 μm to about 70 μm, or from about 15 μm to about 60 μm, or from about 15 μm to about 50 μm, or from about 15 μm to about 40 μm, or from about 15 μm to about 30 μm, or from about 15 μm to about 20 μm, 20 μm to about 1500 μm, or from about 20 μm to about 1000 μm, or from about 20 μm to about 1500 μm, or from about 20 μm to about 900 μm, or from about 20 μm to about 800 μm, or from about 20 μm to about 700 μm, or from about 20 μm to about 600 μm, or from about 20 μm to about 500 μm, or from about 20 μm to about 400 μm, or from about 20 μm to about 300 μm, or from about 20 μm to about 200 μm, or from about 20 μm to about 100 μm, or from about 20 μm to about 90 μm, or from about 20 μm to about 80 μm, or from about 20 μm to about 70 μm, or from about 20 μm to about 60 μm, or from about 20 μm to about 50 μm, or from about 20 μm to about 40 μm, or from about 20 μm to about 30 μm, 30 μm to about 1500 μm, or from about 30 μm to about 1000 μm, or from about 30 μm to about 1500 μm, or from about 30 μm to about 900 μm, or from about 30 μm to about 800 μm, or from about 30 μm to about 700 μm, or from about 30 μm to about 600 μm, or from about 30 μm to about 500 μm, or from about 30 μm to about 400 μm, or from about 30 μm to about 300 μm, or from about 30 μm to about 200 μm, or from about 30 μm to about 100 μm, or from about 30 μm to about 90 μm, or from about 30 μm to about 80 μm, or from about 30 μm to about 70 μm, or from about 30 μm to about 60 μm, or from about 30 μm to about 50 μm, or from about 30 μm to about 40 μm, 40 μm to about 1500 μm, or from about 40 μm to about 1000 μm, or from about 40 μm to about 1500 μm, or from about 40 μm to about 900 μm, or from about 40 μm to about 800 μm, or from about 40 μm to about 700 μm, or from about 40 μm to about 600 μm, or from about 40 μm to about 500 μm, or from about 40 μm to about 400 μm, or from about 40 μm to about 300 μm, or from about 40 μm to about 200 μm, or from about 40 μm to about 100 μm, or from about 40 μm to about 90 μm, or from about 40 μm to about 80 μm, or from about 40 μm to about 70 μm, or from about 40 μm to about 60 μm, or from about 40 μm to about 50 μm, 50 μm to about 1500 μm, or from about 50 μm to about 1000 μm, or from about 50 μm to about 1500 μm, or from about 50 μm to about 900 μm, or from about 50 μm to about 800 μm, or from about 50 μm to about 700 μm, or from about 50 μm to about 600 μm, or from about 50 μm to about 500 μm, or from about 50 μm to about 400 μm, or from about 50 μm to about 300 μm, or from about 50 μm to about 200 μm, or from about 50 μm to about 100 μm, or from about 50 μm to about 90 μm, or from about 50 μm to about 80 μm, or from about 50 μm to about 70 μm, or from about 50 μm to about 60 μm, 60 μm to about 1500 μm, or from about 60 μm to about 1000 μm, or from about 60 μm to about 1500 μm, or from about 60 μm to about 900 μm, or from about 60 μm to about 800 μm, or from about 60 μm to about 700 μm, or from about 60 μm to about 600 μm, or from about 60 μm to about 500 μm, or from about 60 μm to about 400 μm, or from about 60 μm to about 300 μm, or from about 60 μm to about 200 μm, or from about 60 μm to about 100 μm, or from about 60 μm to about 90 μm, or from about 60 μm to about 80 μm, or from about 60 μm to about 70 μm, 70 μm to about 1500 μm, or from about 70 μm to about 1000 μm, or from about 70 μm to about 1500 μm, or from about 70 μm to about 900 μm, or from about 70 μm to about 800 μm, or from about 70 μm to about 700 μm, or from about 70 μm to about 600 μm, or from about 70 μm to about 500 μm, or from about 70 μm to about 400 μm, or from about 70 μm to about 300 μm, or from about 70 μm to about 200 μm, or from about 70 μm to about 100 μm, or from about 70 μm to about 90 μm, or from about 70 μm to about 80 μm, 80 μm to about 1500 μm, or from about 80 μm to about 1000 μm, or from about 80 μm to about 1500 μm, or from about 80 μm to about 900 μm, or from about 80 μm to about 800 μm, or from about 80 μm to about 700 μm, or from about 80 μm to about 600 μm, or from about 80 μm to about 500 μm, or from about 80 μm to about 400 μm, or from about 80 μm to about 300 μm, or from about 80 μm to about 200 μm, or from about 80 μm to about 100 μm, or from about 80 μm to about 90 μm, 90 μm to about 1500 μm, or from about 90 μm to about 1000 μm, or from about 90 μm to about 1500 μm, or from about 90 μm to about 900 μm, or from about 90 μm to about 800 μm, or from about 90 μm to about 700 μm, or from about 90 μm to about 600 μm, or from about 90 μm to about 500 μm, or from about 90 μm to about 400 μm, or from about 90 μm to about 300 μm, or from about 90 μm to about 200 μm, or from about 90 μm to about 100 μm, 100 μm to about 1500 μm, or from about 100 μm to about 1000 μm, or from about 100 μm to about 1500 μm, or from about 100 μm to about 900 μm, or from about 100 μm to about 800 μm, or from about 100 μm to about 700 μm, or from about 100 μm to about 600 μm, or from about 100 μm to about 500 μm, or from about 100 μm to about 400 μm, or from about 100 μm to about 300 μm, or from about 100 μm to about 200 μm, 200 μm to about 1500 μm, or from about 200 μm to about 1000 μm, or from about 200 μm to about 1500 μm, or from about 200 μm to about 900 μm, or from about 200 μm to about 800 μm, or from about 200 μm to about 700 μm, or from about 200 μm to about 600 μm, or from about 200 μm to about 500 μm, or from about 200 μm to about 400 μm, or from about 200 μm to about 300 μm, 300 μm to about 1500 μm, or from about 300 μm to about 1000 μm, or from about 300 μm to about 1500 μm, or from about 300 μm to about 900 μm, or from about 300 μm to about 800 μm, or from about 300 μm to about 700 μm, or from about 300 μm to about 600 μm, or from about 300 μm to about 500 μm, or from about 300 μm to about 400 μm, 400 μm to about 1500 μm, or from about 400 μm to about 1000 μm, or from about 400 μm to about 1500 μm, or from about 400 μm to about 900 μm, or from about 400 μm to about 800 μm, or from about 400 μm to about 700 μm, or from about 400 μm to about 600 μm, or from about 400 μm to about 500 μm, 500 μm to about 1500 μm, or from about 500 μm to about 1000 μm, or from about 500 μm to about 1500 μm, or from about 500 μm to about 900 μm, or from about 500 μm to about 800 μm, or from about 500 μm to about 700 μm, or from about 500 μm to about 600 μm, 600 μm to about 1500 μm, or from about 600 μm to about 1000 μm, or from about 600 μm to about 1500 μm, or from about 600 μm to about 900 μm, or from about 600 μm to about 800 μm, or from about 600 μm to about 700 μm, 700 μm to about 1500 μm, or from about 700 μm to about 1000 μm, or from about 700 μm to about 1500 μm, or from about 700 μm to about 900 μm, or from about 700 μm to about 800 μm, 800 μm to about 1500 μm, or from about 800 μm to about 1000 μm, or from about 800 μm to about 1500 μm, or from about 800 μm to about 900 μm, 900 μm to about 1500 μm, or from about 900 μm to about 1000 μm, 1000 μm to about 1500 μm. Preferable, from about 0.2 μm to about 500 μm.

The thickness of the shell of the microcapsules which may be used in the present invention may vary in the range of 50 nm to 500 μm, and preferably from about 50 nm to about 240 μm. The thickness of the functional surface layer using the post-functionalization method is of several nanometers (1-10 nm). The density of the microcapsules can be as low as 0.001 g/cm³, approximately 1/1000 of the density of most plastics, composites, rubbers, and textiles products. The density of the microcapsule ranges from about as 0.001 g/cm³ to about 1.0 g/cm³, or from about 0.005 g/cm³ to about 1.0 g/cm³, or from about 0.01 g/cm³ to about 1.0 g/cm³, or from about 0.02 g/cm³ to about 1.0 g/cm³, or from about 0.03 g/cm³ to about 1.0 g/cm³, or from about 0.04 g/cm³ to about 1.0 g/cm³, or from about 0.05 g/cm³ to about 1.0 g/cm³, or from about 0.06 g/cm³ to about 1.0 g/cm³, or from about 0.07 g/cm³ to about 1.0 g/cm³, or from about 0.08 g/cm³ to about 1.0 g/cm³, or from about 0.09 g/cm³ to about 1.0 g/cm³, or from about 0.1 g/cm³ to about 1.0 g/cm³, or from about 0.2 g/cm³ to about 1.0 g/cm³, or from about 0.3 g/cm³ to about 1.0 g/cm³, or from about 0.4 g/cm³ to about 1.0 g/cm³, or from about 0.5 g/cm³ to about 1.0 g/cm³, or from about 0.6 g/cm³ to about 1.0 g/cm³, or from about 0.7 g/cm³ to about 1.0 g/cm³, or from about 0.8 g/cm³ to about 1.0 g/cm³, or from about 0.9 g/cm³ to about 1.0 g/cm³, or from about 0.005 g/cm³ to about 1.0 g/cm³, or from about as 0.001 g/cm³ to about 0.9 g/cm³, or from about 0.005 g/cm³ to about 0.9 g/cm³, or from about 0.01 g/cm³ to about 0.9 g/cm³, or from about 0.02 g/cm³ to about 0.9 g/cm³, or from about 0.03 g/cm³ to about 0.9 g/cm³, or from about 0.04 g/cm³ to about 0.9 g/cm³, or from about 0.05 g/cm³ to about 0.9 g/cm³, or from about 0.06 g/cm³ to about 0.9 g/cm³, or from about 0.07 g/cm³ to about 0.9 g/cm³, or from about 0.08 g/cm³ to about 0.9 g/cm³, or from about 0.09 g/cm³ to about 0.9 g/cm³, or from about 0.1 g/cm³ to about 0.9 g/cm³, or from about 0.2 g/cm³ to about 0.9 g/cm³, or from about 0.3 g/cm³ to about 0.9 g/cm³, or from about 0.4 g/cm³ to about 0.9 g/cm³, or from about 0.5 g/cm³ to about 0.9 g/cm³, or from about 0.6 g/cm³ to about 0.9 g/cm³, or from about 0.7 g/cm³ to about 0.9 g/cm³, or from about 0.8 g/cm³ to about 0.9 g/cm³, or from about as 0.001 g/cm³ to about 0.8 g/cm³, or from about 0.005 g/cm³ to about 0.8 g/cm³, or from about 0.01 g/cm³ to about 0.8 g/cm³, or from about 0.02 g/cm³ to about 0.8 g/cm³, or from about 0.03 g/cm³ to about 0.8 g/cm³, or from about 0.04 g/cm³ to about 0.8 g/cm³, or from about 0.05 g/cm³ to about 0.8 g/cm³, or from about 0.06 g/cm³ to about 0.8 g/cm³, or from about 0.07 g/cm³ to about 0.8 g/cm³, or from about 0.08 g/cm³ to about 0.8 g/cm³, or from about 0.09 g/cm³ to about 0.8 g/cm³, or from about 0.1 g/cm³ to about 0.8 g/cm³, or from about 0.2 g/cm³ to about 0.8 g/cm³, or from about 0.3 g/cm³ to about 0.8 g/cm³, or from about 0.4 g/cm³ to about 0.8 g/cm³, or from about 0.5 g/cm³ to about 0.8 g/cm³, or from about 0.6 g/cm³ to about 0.8 g/cm³, or from about 0.7 g/cm³ to about 0.8 g/cm³, or from about as 0.001 g/cm³ to about 0.7 g/cm³, or from about 0.005 g/cm³ to about 0.7 g/cm³, or from about 0.01 g/cm³ to about 0.8 g/cm³, or from about 0.02 g/cm³ to about 0.7 g/cm³, or from about 0.03 g/cm³ to about 0.7 g/cm³, or from about 0.04 g/cm³ to about 0.7 g/cm³, or from about 0.05 g/cm³ to about 0.7 g/cm³, or from about 0.06 g/cm³ to about 0.7 g/cm³, or from about 0.07 g/cm³ to about 0.7 g/cm³, or from about 0.08 g/cm³ to about 0.7 g/cm³, or from about 0.09 g/cm³ to about 0.7 g/cm³, or from about 0.1 g/cm³ to about 0.7 g/cm³, or from about 0.2 g/cm³ to about 0.7 g/cm³, or from about 0.3 g/cm³ to about 0.7 g/cm³, or from about 0.4 g/cm³ to about 0.7 g/cm³, or from about 0.5 g/cm³ to about 0.7 g/cm³, or from about 0.6 g/cm³ to about 0.7 g/cm³, or from about as 0.001 g/cm³ to about 0.6 g/cm³, or from about 0.005 g/cm³ to about 0.6 g/cm³, or from about 0.01 g/cm³ to about 0.8 g/cm³, or from about 0.02 g/cm³ to about 0.6 g/cm³, or from about 0.03 g/cm³ to about 0.6 g/cm³, or from about 0.04 g/cm³ to about 0.6 g/cm³, or from about 0.05 g/cm³ to about 0.6 g/cm³, or from about 0.06 g/cm³ to about 0.6 g/cm³, or from about 0.07 g/cm³ to about 0.6 g/cm³, or from about 0.08 g/cm³ to about 0.6 g/cm³, or from about 0.09 g/cm³ to about 0.6 g/cm³, or from about 0.1 g/cm³ to about 0.6 g/cm³, or from about 0.2 g/cm³ to about 0.6 g/cm³, or from about 0.3 g/cm³ to about 0.6 g/cm³, or from about 0.4 g/cm³ to about 0.6 g/cm³, or from about 0.5 g/cm³ to about 0.6 g/cm³, or from about as 0.001 g/cm³ to about 0.5 g/cm³, or from about 0.005 g/cm³ to about 0.5 g/cm³, or from about 0.01 g/cm³ to about 0.8 g/cm³, or from about 0.02 g/cm³ to about 0.5 g/cm³, or from about 0.03 g/cm³ to about 0.5 g/cm³, or from about 0.04 g/cm³ to about 0.5 g/cm³, or from about 0.05 g/cm³ to about 0.5 g/cm³, or from about 0.06 g/cm³ to about 0.5 g/cm³, or from about 0.07 g/cm³ to about 0.5 g/cm³, or from about 0.08 g/cm³ to about 0.5 g/cm³, or from about 0.09 g/cm³ to about 0.5 g/cm³, or from about 0.1 g/cm³ to about 0.5 g/cm³, or from about 0.2 g/cm³ to about 0.5 g/cm³, or from about 0.3 g/cm³ to about 0.5 g/cm³, or from about 0.4 g/cm³ to about 0.5 g/cm³, or from about as 0.001 g/cm³ to about 0.4 g/cm³, or from about 0.005 g/cm³ to about 0.4 g/cm³, or from about 0.01 g/cm³ to about 0.8 g/cm³, or from about 0.02 g/cm³ to about 0.4 g/cm³, or from about 0.03 g/cm³ to about 0.4 g/cm³, or from about 0.04 g/cm³ to about 0.4 g/cm³, or from about 0.05 g/cm³ to about 0.4 g/cm³, or from about 0.06 g/cm³ to about 0.4 g/cm³, or from about 0.07 g/cm³ to about 0.4 g/cm³, or from about 0.08 g/cm³ to about 0.4 g/cm³, or from about 0.09 g/cm³ to about 0.4 g/cm³, or from about 0.1 g/cm³ to about 0.4 g/cm³, or from about 0.2 g/cm³ to about 0.4 g/cm³, or from about 0.3 g/cm³ to about 0.4 g/cm³, or from about as 0.001 g/cm³ to about 0.3 g/cm³, or from about 0.005 g/cm³ to about 0.3 g/cm³, or from about 0.01 g/cm³ to about 0.8 g/cm³, or from about 0.02 g/cm³ to about 0.3 g/cm³, or from about 0.03 g/cm³ to about 0.3 g/cm³, or from about 0.04 g/cm³ to about 0.3 g/cm³, or from about 0.05 g/cm³ to about 0.3 g/cm³, or from about 0.06 g/cm³ to about 0.3 g/cm³, or from about 0.07 g/cm³ to about 0.3 g/cm³, or from about 0.08 g/cm³ to about 0.3 g/cm³, or from about 0.09 g/cm³ to about 0.3 g/cm³, or from about 0.1 g/cm³ to about 0.3 g/cm³, or from about 0.2 g/cm³ to about 0.3 g/cm³, or from about as 0.001 g/cm³ to about 0.2 g/cm³, or from about 0.005 g/cm³ to about 0.2 g/cm³, or from about 0.01 g/cm³ to about 0.8 g/cm³, or from about 0.02 g/cm³ to about 0.2 g/cm³, or from about 0.03 g/cm³ to about 0.2 g/cm³, or from about 0.04 g/cm³ to about 0.2 g/cm³, or from about 0.05 g/cm³ to about 0.2 g/cm³, or from about 0.06 g/cm³ to about 0.2 g/cm³, or from about 0.07 g/cm³ to about 0.2 g/cm³, or from about 0.08 g/cm³ to about 0.2 g/cm³, or from about 0.09 g/cm³ to about 0.2 g/cm³, or from about 0.1 g/cm³ to about 0.2 g/cm³, or from about as 0.001 g/cm³ to about 0.1 g/cm³, or from about 0.005 g/cm³ to about 0.1 g/cm³, or from about 0.01 g/cm³ to about 0.8 g/cm³, or from about 0.02 g/cm³ to about 0.1 g/cm³, or from about 0.03 g/cm³ to about 0.1 g/cm³, or from about 0.04 g/cm³ to about 0.1 g/cm³, or from about 0.05 g/cm³ to about 0.1 g/cm³, or from about 0.06 g/cm³ to about 0.1 g/cm³, or from about 0.07 g/cm³ to about 0.1 g/cm³, or from about 0.08 g/cm³ to about 0.1 g/cm³, or from about 0.09 g/cm³ to about 0.1 g/cm³, or from about as 0.001 g/cm³ to about 0.09 g/cm³, or from about 0.005 g/cm³ to about 0.09 g/cm³, or from about 0.01 g/cm³ to about 0.8 g/cm³, or from about 0.02 g/cm³ to about 0.09 g/cm³, or from about 0.03 g/cm³ to about 0.09 g/cm³, or from about 0.04 g/cm³ to about 0.09 g/cm³, or from about 0.05 g/cm³ to about 0.09 g/cm³, or from about 0.06 g/cm³ to about 0.09 g/cm³, or from about 0.07 g/cm³ to about 0.09 g/cm³, or from about 0.08 g/cm³ to about 0.09 g/cm³, or from about as 0.001 g/cm³ to about 0.08 g/cm³, or from about 0.005 g/cm³ to about 0.08 g/cm³, or from about 0.01 g/cm³ to about 0.8 g/cm³, or from about 0.02 g/cm³ to about 0.08 g/cm³, or from about 0.03 g/cm³ to about 0.08 g/cm³, or from about 0.04 g/cm³ to about 0.08 g/cm³, or from about 0.05 g/cm³ to about 0.08 g/cm³, or from about 0.06 g/cm³ to about 0.08 g/cm³, or from about 0.07 g/cm³ to about 0.08 g/cm³, or from about as 0.001 g/cm³ to about 0.07 g/cm³, or from about 0.005 g/cm³ to about 0.07 g/cm³, or from about 0.01 g/cm³ to about 0.8 g/cm³, or from about 0.02 g/cm³ to about 0.07 g/cm³, or from about 0.03 g/cm³ to about 0.07 g/cm³, or from about 0.04 g/cm³ to about 0.07 g/cm³, or from about 0.05 g/cm³ to about 0.07 g/cm³, or from about 0.06 g/cm³ to about 0.07 g/cm³, or from about as 0.001 g/cm³ to about 0.06 g/cm³, or from about 0.005 g/cm³ to about 0.06 g/cm³, or from about 0.01 g/cm³ to about 0.8 g/cm³, or from about 0.02 g/cm³ to about 0.06 g/cm³, or from about 0.03 g/cm³ to about 0.06 g/cm³, or from about 0.04 g/cm³ to about 0.06 g/cm³, or from about 0.05 g/cm³ to about 0.06 g/cm³, or from about as 0.001 g/cm³ to about 0.05 g/cm³, or from about 0.005 g/cm³ to about 0.05 g/cm³, or from about 0.01 g/cm³ to about 0.8 g/cm³, or from about 0.02 g/cm³ to about 0.05 g/cm³, or from about 0.03 g/cm³ to about 0.05 g/cm³, or from about 0.04 g/cm³ to about 0.05 g/cm³, or from about as 0.001 g/cm³ to about 0.04 g/cm³, or from about 0.005 g/cm³ to about 0.04 g/cm³, or from about 0.01 g/cm³ to about 0.8 g/cm³, or from about 0.02 g/cm³ to about 0.04 g/cm³, or from about 0.03 g/cm³ to about 0.04 g/cm³, or from about as 0.001 g/cm³ to about 0.03 g/cm³, or from about 0.005 g/cm³ to about 0.03 g/cm³, or from about 0.01 g/cm³ to about 0.03 g/cm³, or from about 0.02 g/cm³ to about 0.03 g/cm³,or from about as 0.001 g/cm³ to about 0.02 g/cm³, or from about 0.005 g/cm³ to about 0.02 g/cm³, or from about 0.01 g/cm³ to about 0.02 g/cm³, or from about as 0.001 g/cm³ to about 0.01 g/cm³, or from about 0.005 g/cm³ to about 0.01 g/cm³, or from about as 0.001 g/cm³ to about 0.005 g/cm³. Preferably, the density is from about 0.01 g/cm³ to about 0.5 g/cm³.

According to an embodiment, the shell comprises from about 0% to about 70% Q3 configuration (i.e. the silicon atoms form siloxane bonds with tree neighbors), and from about 30% to about 100% Q4 configuration (the silicon atoms form siloxane bridges with 4 neighbors). According to another embodiment, the shell comprises from about 40% Q3 configuration and from about 60% Q4 configuration. According to another embodiment, the shell comprises less than about 10% Q3 configuration and more than about 90% Q4 configuration. According to a preferred embodiment the shell comprises 100% Q4 configuration.

According to another embodiment, the shell of the microcapsules which may be used in the present invention may comprise from about 0% to about 60% T2 form silica and from about 40% to about 100% T3 form silica.

According to another embodiment, the shell may comprise combinations of T and Q configurations thereof.

According to another embodiment, a second chemical approach involves nanoscale silica particles being synthesized in situ on the surface of oxidized carbon allotropes using the sol-gel process. Said silica nanoparticles have a diameter of about 5 nm to about 1000 nm, or from about 10 nm to about 1000 nm, or from about 20 nm to about 1000 nm, or from about 30 nm to about 1000 nm, or from about 40 nm to about 1000 nm, or from about 50 nm to about 1000 nm, or from about 60 nm to about 1000 nm, or from about 70 nm to about 1000 nm, or from about 80 nm to about 1000 nm, or from about 90 nm to about 1000 nm, or from about 100 nm to about 1000 nm, or from about 200 nm to about 1000 nm, or from about 300 nm to about 1000 nm, or from about 400 nm to about 1000 nm, or from about 500 nm to about 1000 nm, or from about 600 nm to about 1000 nm, or from about 700 nm to about 1000 nm, or from about 800 nm to about 1000 nm, or from about 900 nm to about 1000 nm, or from about 5 nm to about 900 nm, or from about 10 nm to about 900 nm, or from about 20 nm to about 900 nm, or from about 30 nm to about 900 nm, or from about 40 nm to about 900 nm, or from about 50 nm to about 900 nm, or from about 60 nm to about 900 nm, or from about 70 nm to about 900 nm, or from about 80 nm to about 900 nm, or from about 90 nm to about 900 nm, or from about 100 nm to about 900 nm, or from about 200 nm to about 900 nm, or from about 300 nm to about 900 nm, or from about 400 nm to about 900 nm, or from about 500 nm to about 900 nm, or from about 600 nm to about 900 nm, or from about 700 nm to about 900 nm, or from about 800 nm to about 900 nm, or from about 5 nm to about 800 nm, or from about 10 nm to about 800 nm, or from about 20 nm to about 800 nm, or from about 30 nm to about 800 nm, or from about 40 nm to about 800 nm, or from about 50 nm to about 800 nm, or from about 60 nm to about 800 nm, or from about 70 nm to about 800 nm, or from about 80 nm to about 800 nm, or from about 90 nm to about 800 nm, or from about 100 nm to about 800 nm, or from about 200 nm to about 800 nm, or from about 300 nm to about 800 nm, or from about 400 nm to about 800 nm, or from about 500 nm to about 800 nm, or from about 600 nm to about 800 nm, or from about 700 nm to about 800 nm, or from about 5 nm to about 700 nm, or from about 10 nm to about 700 nm, or from about 20 nm to about 700 nm, or from about 30 nm to about 700 nm, or from about 40 nm to about 700 nm, or from about 50 nm to about 700 nm, or from about 60 nm to about 700 nm, or from about 70 nm to about 700 nm, or from about 80 nm to about 700 nm, or from about 90 nm to about 700 nm, or from about 100 nm to about 700 nm, or from about 200 nm to about 700 nm, or from about 300 nm to about 700 nm, or from about 400 nm to about 700 nm, or from about 500 nm to about 700 nm, or from about 600 nm to about 700 nm, or from about 5 nm to about 600 nm, or from about 10 nm to about 600 nm, or from about 20 nm to about 600 nm, or from about 30 nm to about 600 nm, or from about 40 nm to about 600 nm, or from about 50 nm to about 600 nm, or from about 60 nm to about 600 nm, or from about 70 nm to about 600 nm, or from about 80 nm to about 600 nm, or from about 90 nm to about 600 nm, or from about 100 nm to about 600 nm, or from about 200 nm to about 600 nm, or from about 300 nm to about 600 nm, or from about 400 nm to about 600 nm, or from about 500 nm to about 600 nm, or from about 5 nm to about 500 nm, or from about 10 nm to about 500 nm, or from about 20 nm to about 500 nm, or from about 30 nm to about 500 nm, or from about 40 nm to about 500 nm, or from about 50 nm to about 500 nm, or from about 60 nm to about 500 nm, or from about 70 nm to about 500 nm, or from about 80 nm to about 500 nm, or from about 90 nm to about 500 nm, or from about 100 nm to about 500 nm, or from about 200 nm to about 500 nm, or from about 300 nm to about 500 nm, or from about 400 nm to about 500 nm, or from about 5 nm to about 400 nm, or from about 10 nm to about 400 nm, or from about 20 nm to about 400 nm, or from about 30 nm to about 400 nm, or from about 40 nm to about 400 nm, or from about 50 nm to about 400 nm, or from about 60 nm to about 400 nm, or from about 70 nm to about 400 nm, or from about 80 nm to about 400 nm, or from about 90 nm to about 400 nm, or from about 100 nm to about 400 nm, or from about 200 nm to about 400 nm, or from about 300 nm to about 400 nm, or from about 5 nm to about 300 nm, or from about 10 nm to about 300 nm, or from about 20 nm to about 300 nm, or from about 30 nm to about 300 nm, or from about 40 nm to about 300 nm, or from about 50 nm to about 300 nm, or from about 60 nm to about 300 nm, or from about 70 nm to about 300 nm, or from about 80 nm to about 300 nm, or from about 90 nm to about 300 nm, or from about 100 nm to about 300 nm, or from about 200 nm to about 300 nm, or from about 5 nm to about 200 nm, or from about 10 nm to about 200 nm, or from about 20 nm to about 200 nm, or from about 30 nm to about 200 nm, or from about 40 nm to about 200 nm, or from about 50 nm to about 200 nm, or from about 60 nm to about 200 nm, or from about 70 nm to about 200 nm, or from about 80 nm to about 200 nm, or from about 90 nm to about 200 nm, or from about 100 nm to about 200 nm, or from about 5 nm to about 100 nm, or from about 10 nm to about 100 nm, or from about 20 nm to about 100 nm, or from about 30 nm to about 100 nm, or from about 40 nm to about 100 nm, or from about 50 nm to about 100 nm, or from about 60 nm to about 100 nm, or from about 70 nm to about 100 nm, or from about 80 nm to about 100 nm, or from about 90 nm to about 100 nm, or from about 5 nm to about 90 nm, or from about 10 nm to about 90 nm, or from about 20 nm to about 90 nm, or from about 30 nm to about 90 nm, or from about 40 nm to about 90 nm, or from about 50 nm to about 90 nm, or from about 60 nm to about 90 nm, or from about 70 nm to about 90 nm, or from about 80 nm to about 90 nm, or from about 5 nm to about 80 nm, or from about 10 nm to about 80 nm, or from about 20 nm to about 80 nm, or from about 30 nm to about 80 nm, or from about 40 nm to about 80 nm, or from about 50 nm to about 80 nm, or from about 60 nm to about 80 nm, or from about 70 nm to about 80 nm, or from about 5 nm to about 70 nm, or from about 10 nm to about 70 nm, or from about 20 nm to about 70 nm, or from about 30 nm to about 70 nm, or from about 40 nm to about 70 nm, or from about 50 nm to about 70 nm, or from about 60 nm to about 70 nm, or from about 5 nm to about 60 nm, or from about 10 nm to about 60 nm, or from about 20 nm to about 60 nm, or from about 30 nm to about 60 nm, or from about 40 nm to about 60 nm, or from about 50 nm to about 60 nm, or from about 5 nm to about 50 nm, or from about 10 nm to about 50 nm, or from about 20 nm to about 50 nm, or from about 30 nm to about 50 nm, or from about 40 nm to about 50 nm, or from about 5 nm to about 40 nm, or from about 10 nm to about 40 nm, or from about 20 nm to about 40 nm, or from about 30 nm to about 40 nm, or from about 5 nm to about 30 nm, or from about 10 nm to about 30 nm, or from about 20 nm to about 30 nm, or from about 5 nm to about 20 nm, or from about 10 nm to about 20 nm, or from about 5 nm to about 10 nm, and preferably from 10 to 100 nm. The in situ synthesis of silica nanoparticles is performed by dispersing pre-oxidized carbon allotropes in a polar solvent (water, alcohols, DMF, DMSO, etc.), followed by subsequent additions of an alkoxysilane (methoxysilane, an ethoxysilane, a propoxysilane, an isopropoxysilane, an aryloxysilane, tetramethoxysilane (TMOS), tetraethoxysilane (TEOS), tetrapropoxysilane (TPOS) or a functional trimethoxy, triethoxysilane, tripropoxysilane including aminopropylsilane, aminoethylaminopropylsilane, vinyltrimethoxysilane, 3-chloropropyltriethoxysilane, 3-glycidoxypropyltrimethoxysilane, methacryloyloxypropyltrimethoxysilane, phenyltriethoxysilane, phenyltrimethoxysilane, glycidoxypropoxyltrimethoxysilane, glycidoxypropyltriethoxysilane, mercaptopropyltriethoxysilane, mercaptopropyltrimethoxysilane, aminopropyltrimethoxysilane, 3-aminopropyltriethoxysilane, 3-(2-aminoethylamino)propyltrimethoxysilane, 3-[2-(2-aminoethylamino)ethylamino]propyltrimethoxysilane, [2(cyclohexenyl)ethyl]triethoxysilane, vinyltrimethoxysilane, vinyltriethoxysilane or a mixture of any two or more of the above) and a catalyst for sol-gel reaction (chloridric acid, sulfuric acid, ammonia, sodium hydroxide, etc.) under stirring or ultrasonication. This affords various hybrid materials with silica nanoparticles decorating the surface of carbon allotropes (graphene, graphite, carbon nanofibers, carbon nanotubes, etc.). The covalent attachment is possible due to the presence on oxidized carbon allotropes of hydroxyl groups and the conversion of carbonyl groups (C═O) to a Si—O—C bonding after the reaction with an alkoxysilane.

### Physical Processes

According to another embodiment of the present invention, silica-carbon allotrope composites materials may also be prepared using a physical process. Following this approach, the carbon allotropes are directly formed using a plasma deposition process in presence of silica microspheres.

Thermal plasmas, generated by DC (direct current) arc or inductively coupled RF (Radio Frequency) discharge are well-known and powerful processes in the production of carbon nanostructures. Using these techniques, various carbon allotropes including graphene, carbon nanofibers, carbon nanotubes, etc. have been successfully synthesized for two decades (Nature, 1991, 354, 56-58; Science, 1998, 282, 1105-1107; Appl. Phys. Lett., 2000, 77, 830-832). Moreover, with plasma treatment, heteroatoms (e.g. nitrogen, sulfur) have been successfully introduced in carbon nanomaterials in order to modify their electronic and physico-chemical properties (Carbon, 2010, 48, 255-259; Plasma Chem. Plasma Process, 2011, 31, 393-403; International patent No WO2014000108 A1). In this invention, a focus has been paid on the development of new composite materials made of silica microparticles and carbon nanostructures, taking advantage of the versatility of the RF plasma deposition process.

According to an embodiment, the plasma can be produced using an inductively coupled radio-frequency torch operated using powers in the range of 1 to 50 kW, or from about 5 to 50 kW, or from about 10 to 50 kW, or from about 15 to about 50 kW, or from about 20 to 50 kW, or from about 25 to about 50 kW, or from about 30 to about 50 kW, or from about 35 to about 50 kW, or from about 40 to about 50 kW, or from about 45 to about 50 kW, or from about 5 to 45 kW, or from about 10 to 45 kW, or from about 15 to about 45 kW, or from about 20 to 45 kW, or from about 25 to about 45 kW, or from about 30 to about 45 kW, or from about 35 to about 45 kW, or from about 40 to about 45 kW, or from about 5 to 40 kW, or from about 10 to 40 kW, or from about 15 to about 40 kW, or from about 20 to 40 kW, or from about 25 to about 40 kW, or from about 30 to about 40 kW, or from about 35 to about 40 kW, or from about 5 to 35 kW, or from about 10 to 35 kW, or from about 15 to about 35 kW, or from about 20 to 35 kW, or from about 25 to about 35 kW, or from about 30 to about 35 kW, or from about 5 to 30 kW, or from about 10 to 30 kW, or from about 15 to about 30 kW, or from about 20 to 30 kW, or from about 25 to about 30 kW, or from about 5 to 25 kW, or from about 10 to 25 kW, or from about 15 to about 25 kW, or from about 20 to 25 kW, or from about 5 to 20 kW, or from about 10 to 20 kW, or from about 15 to about 20 kW, or from about 5 to 15 kW, or from about 10 to 15 kW, or from about 5 to 10 kW, preferably in the range of 5 to 20 kW. The carbon precursor for the synthesis of carbon allotropes can be any carbon source able to be vaporized under the temperature and pressure reaction conditions of the present invention. The carbon source can be chosen from hydrocarbons including aromatic hydrocarbons (benzene, toluene, xylene, etc.), aliphatic hydrocarbons (methane, propane, hexane, heptanes, etc.), branched hydrocarbons (ethers, ketones, alcohols, etc.), chlorinated hydrocarbons (chloroform, methylene chloride, trichloroethylene, etc.) and mixtures thereof. The carbon source may be liquid or gaseous at room temperature and atmospheric pressure, although it is typically used in the plasma deposition process in vapor form, as the central plasmagenic gas. According to another embodiment, the central plasmagenic gas is preferably methane. The central plasmagenic gas can be injected in the chamber at a pressure of in the range of 172.37 kPa to about 517.11 kPa [25 to 75 pound per square inch (psi)], or from about 206.84 kPa to about 517.11 kPa, or from about 241.32 kPa to about 517.11 kPa, or from about 275.79 kPa to about 517.11 kPa, or from about 310.26 kPa to about 517.11 kPa, or from about 344.74 kPa to about 517.11 kPa, or from about 379.21 kPa to about 517.11 kPa, or from about 413.69 kPa to about 517.11 kPa, or from about 448.16 kPa to about 517.11 kPa, or from about 482.63 kPa to about 517.11 kPa, or from about 172.37 kPa to about 482.63 kPa, or from about 206.84 kPa to about 482.63 kPa, or from about 241.32 kPa to about 482.63 kPa, or from about 275.79 kPa to about 482.63 kPa, or from about 310.26 kPa to about 482.63 kPa, or from about 344.74 kPa to about 482.63 kPa, or from about 379.21 kPa to about 482.63 kPa, or from about 413.69 kPa to about 482.63 kPa, or from about 448.16 kPa to about 482.63 kPa, or from about 172.37 kPa to about 448.16 kPa, or from about 206.84 kPa to about 448.16 kPa, or from about 241.32 kPa to about 448.16 kPa, or from about 275.79 kPa to about 448.16 kPa, or from about 310.26 kPa to about 448.16 kPa, or from about 344.74 kPa to about 448.16 kPa, or from about 379.21 kPa to about 448.16 kPa, or from about 413.69 kPa to about 448.16 kPa, or from about 172.37 kPa to about 413.69 kPa, or from about 206.84 kPa to about 413.69 kPa, or from about 241.32 kPa to about 413.69 kPa, or from about 275.79 kPa to about 413.69 kPa, or from about 310.26 kPa to about 413.69 kPa, or from about 344.74 kPa to about 413.69 kPa, or from about 379.21 kPa to about 413.69 kPa, or from about 172.37 kPa to about 379.21 kPa, or from about 206.84 kPa to about 379.21 kPa, or from about 241.32 kPa to about 379.21 kPa, or from about 275.79 kPa to about 379.21 kPa, or from about 310.26 kPa to about 379.21 kPa, or from about 344.74 kPa to about 379.21 kPa, or from about 172.37 kPa to about 344.74 kPa, or from about 206.84 kPa to about 344.74 kPa, or from about 241.32 kPa to about 344.74 kPa, or from about 275.79 kPa to about 344.74 kPa, or from about 310.26 kPa to about 344.74 kPa, or from about 172.37 kPa to about 310.26 kPa, or from about 206.84 kPa to about 310.26 kPa, or from about 241.32 kPa to about 310.26 kPa, or from about 275.79 kPa to about 310.26 kPa, or from about 172.37 kPa to about 275.79 kPa, or from about 206.84 kPa to about 275.79 kPa, or from about 241.32 kPa to about 275.79 kPa, or from about 172.37 kPa to about 241.32 kPa, or from about 206.84 kPa to about 241.32 kPa, or from about 172.37 kPa to about 206.84 kPa, and preferably from about 275.79 kPa to about 413.69 kPa (from about 40 to about 60 psi). The flow rate of the central plasmagenic gas can range from 0.1 to 1.5 standard litres per minute (slpm), or from about 0.2 to 1.5 slpm, or from about 0.3 to 1.5 slpm, or from about 0.4 to 1.5 slpm, or from about 0.5 to 1.5 slpm, or from about 0.6 to 1.5 slpm, or from about 0.7 to 1.5 slpm, or from about 0.8 to 1.5 slpm, or from about 0.9 to 1.5 slpm, or from about 1.0 to 1.5 slpm, or from about 1.1 to 1.5 slpm, or from about 1.2 to 1.5 slpm, or from about 1.3 to 1.5 slpm, or from about 1.4 to 1.5 slpm, or from about 0.2 to 1.4 slpm, or from about 0.3 to 1.4 slpm, or from about 0.4 to 1.4 slpm, or from about 0.5 to 1.4 slpm, or from about 0.6 to 1.4 slpm, or from about 0.7 to 1.4 slpm, or from about 0.8 to 1.4 slpm, or from about 0.9 to 1.4 slpm, or from about 1.0 to 1.4 slpm, or from about 1.1 to 1.4 slpm, or from about 1.2 to 1.4 slpm, or from about 1.3 to 1.4 slpm, or from about 0.2 to 1.3 slpm, or from about 0.3 to 1.3 slpm, or from about 0.4 to 1.3 slpm, or from about 0.5 to 1.3 slpm, or from about 0.6 to 1.3 slpm, or from about 0.7 to 1.3 slpm, or from about 0.8 to 1.3 slpm, or from about 0.9 to 1.3 slpm, or from about 1.0 to 1.3 slpm, or from about 1.1 to 1.3 slpm, or from about 1.2 to 1.3 slpm, or from about 0.2 to 1.2 slpm, or from about 0.3 to 1.2 slpm, or from about 0.4 to 1.2 slpm, or from about 0.5 to 1.2 slpm, or from about 0.6 to 1.2 slpm, or from about 0.7 to 1.2 slpm, or from about 0.8 to 1.2 slpm, or from about 0.9 to 1.2 slpm, or from about 1.0 to 1.2 slpm, or from about 1.1 to 1.2 slpm, or from about 0.2 to 1.1 slpm, or from about 0.3 to 1.1 slpm, or from about 0.4 to 1.1 slpm, or from about 0.5 to 1.1 slpm, or from about 0.6 to 1.1 slpm, or from about 0.7 to 1.1 slpm, or from about 0.8 to 1.1 slpm, or from about 0.9 to 1.1 slpm, or from about 1.0 to 1.1 slpm, or from about 0.2 to 1.0 slpm, or from about 0.3 to 1.0 slpm, or from about 0.4 to 1.0 slpm, or from about 0.5 to 1.0 slpm, or from about 0.6 to 1.0 slpm, or from about 0.7 to 1.0 slpm, or from about 0.8 to 1.0 slpm, or from about 0.9 to 1.0 slpm, or from about 0.2 to 0.9 slpm, or from about 0.3 to 0.9 slpm, or from about 0.4 to 0.9 slpm, or from about 0.5 to 0.9 slpm, or from about 0.6 to 0.9 slpm, or from about 0.7 to 0.9 slpm, or from about 0.8 to 0.9 slpm, or from about 0.2 to 0.8 slpm, or from about 0.3 to 0.8 slpm, or from about 0.4 to 0.8 slpm, or from about 0.5 to 0.8 slpm, or from about 0.6 to 0.8 slpm, or from about 0.7 to 0.8 slpm, or from about 0.2 to 0.7 slpm, or from about 0.3 to 0.7 slpm, or from about 0.4 to 0.7 slpm, or from about 0.5 to 0.7 slpm, or from about 0.6 to 0.7 slpm, or from about 0.2 to 0.6 slpm, or from about 0.3 to 0.6 slpm, or from about 0.4 to 0.6 slpm, or from about 0.5 to 0.6 slpm, or from about 0.2 to 0.5 slpm, or from about 0.3 to 0.5 slpm, or from about 0.4 to 0.5 slpm, or from about 0.2 to 0.4 slpm, or from about 0.3 to 0.4 slpm, or from about 0.2 to 0.3 slpm, and preferably from 0.4 to 0.9 slpm.

The sheath gas, which is typically an inert gas (nitrogen, argon, etc), more preferably argon, allow to constraint the trajectory of the central gas during the deposition process. Indeed, no carbon allotrope can be formed if the central plasmagenic gas is introduced in the sheath gas port. The sheath gas can be injected at a pressure of 172.37 kPa to about 517.11 kPa [25 to 75 pound per square inch (psi)], or from about 206.84 kPa to about 517.11 kPa, or from about 241.32 kPa to about 517.11 kPa, or from about 275.79 kPa to about 517.11 kPa, or from about 310.26 kPa to about 517.11 kPa, or from about 344.74 kPa to about 517.11 kPa, or from about 379.21 kPa to about 517.11 kPa, or from about 413.69 kPa to about 517.11 kPa, or from about 448.16 kPa to about 517.11 kPa, or from about 482.63 kPa to about 517.11 kPa, or from about 172.37 kPa to about 482.63 kPa, or from about 206.84 kPa to about 482.63 kPa, or from about 241.32 kPa to about 482.63 kPa, or from about 275.79 kPa to about 482.63 kPa, or from about 310.26 kPa to about 482.63 kPa, or from about 344.74 kPa to about 482.63 kPa, or from about 379.21 kPa to about 482.63 kPa, or from about 413.69 kPa to about 482.63 kPa, or from about 448.16 kPa to about 482.63 kPa, or from about 172.37 kPa to about 448.16 kPa, or from about 206.84 kPa to about 448.16 kPa, or from about 241.32 kPa to about 448.16 kPa, or from about 275.79 kPa to about 448.16 kPa, or from about 310.26 kPa to about 448.16 kPa, or from about 344.74 kPa to about 448.16 kPa, or from about 379.21 kPa to about 448.16 kPa, or from about 413.69 kPa to about 448.16 kPa, or from about 172.37 kPa to about 413.69 kPa, or from about 206.84 kPa to about 413.69 kPa, or from about 241.32 kPa to about 413.69 kPa, or from about 275.79 kPa to about 413.69 kPa, or from about 310.26 kPa to about 413.69 kPa, or from about 344.74 kPa to about 413.69 kPa, or from about 379.21 kPa to about 413.69 kPa, or from about 172.37 kPa to about 379.21 kPa, or from about 206.84 kPa to about 379.21 kPa, or from about 241.32 kPa to about 379.21 kPa, or from about 275.79 kPa to about 379.21 kPa, or from about 310.26 kPa to about 379.21 kPa, or from about 344.74 kPa to about 379.21 kPa, or from about 172.37 kPa to about 344.74 kPa, or from about 206.84 kPa to about 344.74 kPa, or from about 241.32 kPa to about 344.74 kPa, or from about 275.79 kPa to about 344.74 kPa, or from about 310.26 kPa to about 344.74 kPa, or from about 172.37 kPa to about 310.26 kPa, or from about 206.84 kPa to about 310.26 kPa, or from about 241.32 kPa to about 310.26 kPa, or from about 275.79 kPa to about 310.26 kPa, or from about 172.37 kPa to about 275.79 kPa, or from about 206.84 kPa to about 275.79 kPa, or from about 241.32 kPa to about 275.79 kPa, or from about 172.37 kPa to about 241.32 kPa, or from about 206.84 kPa to about 241.32 kPa, or from about 172.37 kPa to about 206.84 kPa, and preferably from about 275.79 kPa to about 413.69 kPa (from about 40 to about 60 psi) with a flow rate of 1-50 slpm, more preferably 6-35 slpm.

As used herein, the term carrier gas is intended to mean the gas formed between the central gas of carbon or other precursors, and the sheath gas. The carrier gas is typically composed of a hydrocarbon vapor (vapor of aliphatic, cyclic or branched hydrocarbons)(but which may also contain other precursors, such as sulfur or nitrogen-containing precursors), preferably methane, diluted in an inert gas, preferably argon. Concentration of hydrocarbon in the carrier gas can be between about 1.7 to about 8% v/v, or from about 2% to about 8%, or from about 3% to about 8%, or from about 4% to about 8%, or from about 5% to about 8%, or from about 6% to about 8%, or from about 7% to about 8%, or from about 1.7% to about 7%, or from about or from about 2% to about 7%, or from about 3% to about 7%, or from about 4% to about 7%, or from about 5% to about 7%, or from about 6% to about 7%, or from about 1.7% to about 6%, or from about or from about 2% to about 6%, or from about 3% to about 6%, or from about 4% to about 6%, or from about 5% to about 6%, or from about 1.7% to about 5%, or from about or from about 2% to about 5%, or from about 3% to about 5%, or from about 4% to about 5%, or from about 1.7% to about 4%, or from about or from about 2% to about 4%, or from about 3% to about 4%, or from about 1.7% to about 3%, or from about or from about 2% to about 3%, or from about 1.7% to about 2%, and preferably in the range of 4-8% (v/v).

Silica microcapsules which are described in as described in International patent Application publication No. WO2013/078551 may be typically used in solution. This solution can be composed of water, organic solvents (polar or non-polar solvents), vegetable oils and combinations thereof. Synthesis of carbon allotropes and subsequent in situ deposition on microparticles occur at an operating pressure of from about 13.33 kPa to about 61.33 kPa (100-460 Torr), or from about 26.66 kPa to about 61.33 kPa, or from about 40. 00 kPa to about 61.33 kPa, or from about 53.33 kPa to about 61.33 kPa, or from about 13.33 kPa to about 53.33 kPa, or from about 26.66 kPa to about 53.33 kPa, or from about 40. 00 kPa to about 53.33 kPa, or from about 13.33 kPa to about 40. 00 kPa, or from about 26.66 kPa to about 40. 00 kPa, or from about 13.33 kPa to about 26.66 kPa,

According to another embodiment, the operating pressure is preferably in the range of from about 24 kPa to about 42.66 kPa (180-320 Torr), or from about 26.66 kPa to about 42.66 kPa, or from about 29.33 kPa to about 42.66 kPa, or from about 32.00 kPa to about 42.66 kPa, or from about 34.66 kPa to about 42.66 kPa, or from about 37.33 kPa to about 42.66 kPa, or from about 40. 00 kPa to about 42.66 kPa, or from about 24 kPa to about 40. 00 kPa, or from about 26.66 kPa to about 40. 00 kPa, or from about 29.33 kPa to about 40. 00 kPa, or from about 32.00 kPa to about 40. 00 kPa, or from about 34.66 kPa to about 40. 00 kPa, or from about 37.33 kPa to about 40. 00 kPa, or from about 24 kPa to about 37.33 kPa, or from about 26.66 kPa to about 37.33 kPa, or from about 29.33 kPa to about 37.33 kPa, or from about 32.00 kPa to about 37.33 kPa, or from about 34.66 kPa to about 37.33 kPa, or from about 24 kPa to about 34.66 kPa, or from about 26.66 kPa to about 34.66 kPa, or from about 29.33 kPa to about 34.66 kPa, or from about 32.00 kPa to about 34.66 kPa, or from about 24 kPa to about 32.00 kPa, or from about 26.66 kPa to about 32.00 kPa, or from about 29.33 kPa to about 32.00 kPa, or from about 24 kPa to about 29.33 kPa, or from about 26.66 kPa to about 29.33 . kPa, or from about 24 kPa to about 26.66 kPa.

The deposition of the carbon allotropes on the silica microparticles occur in a reactor by injecting a suspension in the vicinity were the carbon allotrope is formed. It is possible to control the level of interaction between the silica microparticles and the plasma torch by controlling the injection point of the silica microparticles suspension in order to favor the interaction between the silica microparticles while preserving their mechanical and chemical integrity. Three configurations are possible for the in situ deposition of carbon allotropes on silica microparticles (Scheme 2). The first configuration consists of a main and an auxiliary tubular reactor in which injection is carried out in the probe, and injected concentric to the plasma torch. In a second configuration, the suspension of microparticles is injected through the top flange of the main reactor and is allowed to partly interact with the skirt of the torch. In the third configuration, the suspension of microparticles is injected from the bottom flange and into the periphery of the plume, at the bottom part of the main reactor.

According to another embodiment of the present invention, the silica microspheres can be mixed or bound to carbon allotropes functionalized with sulfur-, oxygen-, nitrogen-, or halogen-containing functional groups. These functional groups can be added to the carbon allotrope during growth in the plasma reactor by co-introducing oxygen, nitrogen, halogen or sulfur precursors or combination thereof. Nitrogen, oxygen, halogen or sulfur precursors can be in the solid, liquid or gaseous phase or a combination thereof. According to an embodiment, the nitrogen-containing functional group may be an amine group, a ketimine group, an aldimine group, an imide group, an azide group, an azo group, a cyanate group, an isocyanate group, a nitrate group, a nitrile group, a nitrite group, a nitroso group, a nitro group, a pyridyl group and a combination thereof. According to an embodiment, the sulfur-containing functional group may be an sulfhydryl group, a sulfide group, a disulfide group, a sulfinyl group, a sulfonyl group, a sulfo group, a thiocyanate group, carbonothioyl group, carbonothioyl group and a combination thereof. According to an embodiment, the oxygen-containing functional group may be an hydroxyl group, a carbonyl group, an aldehyde group, a carboxylate group, a carboxyl group, an ester group, a methoxy group, a peroxy group, an ether group, a carbonate ester and a combination thereof. According to an embodiment, the halogen-containing functional group is a fluoro, a chloro, a bromo, an iodo and a combination thereof.

The nitrogen, oxygen, halogen or sulfur precursor is injected using the plasma probe and can be mixed either with the carbon precursor or with the carrier gas. The nitrogen, oxygen, halogen or sulfur precursor is injected at a rate between about 0.1 and about 10 slpm, or from about 0.1 and about 9 slpm, or from about 0.1 and about 8 slpm, or from about 0.1 and about 7 slpm, or from about 0.1 and about 6 slpm, or from about 0.1 and about 5 slpm, or from about 0.1 and about 4 slpm, or from about 0.1 and about 3 slpm, or from about 0.1 and about 2 slpm, or from about 0.1 and about 1 slpm, about 1 and about 10 slpm, or from about 1 and about 9 slpm, or from about 1 and about 8 slpm, or from about 1 and about 7 slpm, or from about 1 and about 6 slpm, or from about 1 and about 5 slpm, or from about 1 and about 4 slpm, or from about 1 and about 3 slpm, or from about 1 and about 2 slpm, about 2 and about 10 slpm, or from about 2 and about 9 slpm, or from about 2 and about 8 slpm, or from about 2 and about 7 slpm, or from about 2 and about 6 slpm, or from about 2 and about 5 slpm, or from about 2 and about 4 slpm, or from about 2 and about 3 slpm, about 3 and about 10 slpm, or from about 3 and about 9 slpm, or from about 3 and about 8 slpm, or from about 3 and about 7 slpm, or from about 3 and about 6 slpm, or from about 3 and about 5 slpm, or from about 3 and about 4 slpm, about 4 and about 10 slpm, or from about 4 and about 9 slpm, or from about 4 and about 8 slpm, or from about 4 and about 7 slpm, or from about 4 and about 6 slpm, or from about 4 and about 5 slpm, about 5 and about 10 slpm, or from about 5 and about 9 slpm, or from about 5 and about 8 slpm, or from about 5 and about 7 slpm, or from about 5 and about 6 slpm, about 6 and about 10 slpm, or from about 6 and about 9 slpm, or from about 6 and about 8 slpm, or from about 6 and about 7 slpm, about 7 and about 10 slpm, or from about 7 and about 9 slpm, or from about 7 and about 8 slpm, about 8 and about 10 slpm, or from about 8 and about 9 slpm, about 9 and about 10 slpm, and preferably between 1 and 6 slpm. The decomposition of the precursor can be assisted by the presence of reducing gas, such as H₂, NH₃, H₂O, CO co-injected with the carbon, nitrogen halogen or sulfur precursor at a concentration between 0 and 90% v/v (volume of reducing gas/volume of nitrogen or sulfur precursor).

### Potential Applications

According to an embodiment, the obtained silica-carbon allotrope composite materials may be used in numerous applications. They may be incorporated in various matrices including plastics, composites, rubbers, adhesives or silicones for applications in electronics, solar cells, electrostatic charge-dissipating coatings, thermally conductive materials, electrically conductive materials, low CTE (coefficient of thermal expansion) materials, etc. Moreover, their ultra-low densities allow their use as weight-reducing fillers for polymers and composites materials.

Carbon allotrope-silica hybrid materials of the present invention can also be useful for adsorption and immobilization applications. Indeed, due the ultra-high specific area of carbon allotropes (theoretical value of 2630 m²/g for graphene for example), carbon allotrope-silica microparticles may be used as high-performance sorbents able to give rise to high densities of attached analyte molecules. In addition, the presence of functional groups on the surface of silica microcapsules or silica-carbon allotrope microparticles may serve for the immobilization of various chemical or biological species through covalent or non-covalent bonds.

For more specific applications, hybrid materials obtained from hollow silica particles according to the present invention can be loaded with functional species including fluorescent molecules, magnetic molecules, catalyst molecules, small and macro biological molecules. For instance, since silica and carbon allotropes have low magnetic susceptibility, the incorporation of magnetic nanoparticles (magnetite, maghemite, etc.) in the core of silica capsules may be helpful for those applications requiring magnetic properties.

### EXAMPLE OF APPLICATIONS

Use of Silica-Carbon Allotrope Microparticles as Thermally Conductive and/or Electrically Conductive Fillers for Polymers and Polymer-Based Composites

The silica-carbon allotrope microparticles of the present invention may be introduced into plastics, rubbers or polymer-based composites, or products in their processing stages. They can be dispersed in solution or in bulk into the final products throughout or in parts thereof. With regard to the thermal and electrical conductivities feature, the silica-carbon allotrope microparticles of the present invention may be excellent thermally and/or electrically conductive fillers for many polar and non-polar polymer resins and polymer blends, including low, medium and high density polyethylene (LD or HDPE), polypropylene (PP), polystyrene (PS), polycarbonate (PC), polyurethane (PU), polybutadiene (PB), polyethylene terephthalate (PET), polybutylene terephthalate (PBT), polyoxymethylene (POM), polymethacrylate (PMA), poly(methyl methacrylate) (PMMA), nylon, polyvinyl chloride) (PVC), Acrylonitrile butadiene styrene (ABS), polylactide (PLA), polyvinylidene chloride, and polyether ether ketone (PEK), etc. For instance, these silica-carbon allotrope composite materials can be very interesting for applications requiring materials with high thermal conductivity, such as thermal interlace materials (TIMs) used in semiconductors.

Use of Silica Microcapsules and Silica-Carbon Allotrope Composite Microparticles as Carriers for Microorganisms and Enzymes

According to another applications, silica microcapsules obtained from the process described in International patent Application publication No. WO2013/078551 or the above mentioned silica-carbon allotrope composite microparticles can be used as carriers for microorganisms and enzymes. The obtained microparticles can be used in chemical and biochemical industries (bioorganic synthesis of fine and commodity chemicals) and for biological applications such as, but not limited to, biological wastewater treatment, industrial fermentation and enzymes uses, pharmaceutical fermentation and enzymes uses, biogas production, fermentation and enzymes use in the food industry, bio-filtration of gases, etc.

According to embodiments of the present invention, carriers for cells such as prokaryotic cells (i.e. from microorganisms), as well as eukaryotic cell derived from multicellular organisms, enzymes, and viruses, are defined as particles on which microorganisms, enzymes or viral particles may be immobilized. Such carriers may also be referred to as, but not limited to, immobilization support or immobilization media. The term immobilization includes adsorption, physisorption, covalent immobilization and biofilm supported immobilization.

According to an embodiment, suitable bacterial cells may be chosen from the following phyla: Acidobacteria, Actinobacteria, Aquificae , Bacteroidetes, Caldiserica, Chlamydiae, Chlorobi, Chloroflexi, Chrysiogenetes, Cyanobacteria, Deferribacteres, Deinococcus-Thermus, Dictyoglomi, Elusimicrobia, Fibrobacteres, Firmicutes, Fusobacteria, Gemmatimonadetes, Lentisphaerae, Nitrospira, Planctomycetes, Proteobacteria, Spirochaetes, Synergistetes, Tenericutes, Thermodesulfobacteria, Thermotogae, Verrucomicrobia. More specifically, suitable species which can be used with the present invention may be chosen from but not limited to the following genera: Pseudomonas, Rhodopseudomonas, Acinetobacter, Mycobacterium, Corynebacterium, Arthrobacterium, Bacillius, Flavorbacterium, Nocardia, Achromobacterium, Alcaligenes, Vibrio, Azotobacter, Beijerinckia, Xanthomonas. Nitrosomonas, Nitrobacter, Methylosinus, Methylococcus, Actinomycetes and Methylobacter, etc. Suitable fungi such as yeast can be chosen from but not limited to the following genera: Saccaromyces, Pichia, Brettanomyces, Yarrowia, Candida, Schizosaccharomyces, Torulaspora, Zygosaccharomyces, etc. Suitable fungi from the following phyla can be chosen: Blastocladiomycota, Chytridiomycota, Glomeromycota, Microsporidia, Neocallimastigomycota, Ascomycota, Basidiomycota. More specifically, suitable fungi such as mold can be chosen from but not limited to the following genera: Aspergillus, Rhizopus, Trichoderma, Monascus, Penicillium, Fusarium, Geotrichum, Neurospora, Rhizomucor, and Tolupocladium. Suitable fungi can also be chosen from the mushroom clade.

According to an embodiment, suitable protozoan may be chosen from the following phyla: Percolozoa, Euglenozoa, Ciliophora, Mioza, Dinoza, Apicomplexa, Opalozoa, Mycetozoa, Radiozoa, Heliozoa, Rhizopoda, Neosarcodina, Reticulosa, Choanozoa, Myxosporida, Haplosporida, Paramyxia

Microorganisms are not limited to bacteria, and fungi, but may be extended to include other known microorganisms such as algae, and protozoans. Microorganisms include all states of their living cycle, including the sporulation state.

Eukaryotic cells also include, but are not limited to insect cells such as Drosophila S2 cells, Spodoptera frugiperda Sf21 and Sf9 cells, and the likes. Also included are plant cells, and mammalian cells, such as CHO cells, HeLa cells, HEK293 cells, and the likes.

Suitable enzymes can be chosen from the following classes, but not limited to: oxidoreductases, transferases, hydrolases, lyases, isomerases, ligases, polymerases. Example are amylase, lipase, protease, esterase, etc.

Silica microcapsules and silica-carbon allotrope composite microparticles of the present invention are suitable for biological reactor such as, but not limited to, fermentation batch reactor, enzymatic batch reactor, nitrification reactor, digester reactor, membrane bioreactor (MBR), moving bed bioreactor (MBBR), fluid bed reactor (FBR), continuous stirred reactor (CSTR), plug flow reactor (PFR) and sequential batch reactor (SBR). They may also be used in upflow or downflow fixed film system. Reactor and bioprocess can be run under anaerobic and aerobic conditions.

In the biological treatment of wastewater for example, different microorganisms with specialized metabolic capabilities can be used to adhere to the microparticles and thus serve as biocatalysts for the biodegradation of target compounds. During this biodegradation process, parameters such as pH, oxygenation, nutrient concentrations, temperature, salinity, etc. may be adapted to provide better conditions for the growth of microorganisms.

Nutrients can be introduced into the reactor to enhance the growth of microorganisms and to thus catalyze the biodegradation of contaminants process. According to an embodiment, nutrients may be loaded in the silica microcapsules prior to use as microorganisms carrier. Wastewater contaminants which can be degraded by microorganisms according to the present invention include but are not limited to aromatic compounds, hydrocarbon compounds, halogenated organic compounds, phenolic compounds, alcohol compounds, ketone compounds, carboxylic acid compounds, ammonia containing compounds, nitrate compounds, nitrogenous organic compounds, aldehyde compounds, ether compounds, ester compounds, organosulfur compounds, naphtenic acid compounds, organophosphorus compounds and combinations thereof.

Silica microcapsules and silica-carbon allotrope composite microparticles of the present invention are suitable for agriculture used as bioinnoculant and biofertiliser. Similarly in water treatment and in industrial biotechnology, silica microcapsules and silica-carbon allotrope composite microparticles are used to immobilize microorganisms.

Example of applications and benefits for cells immobilization are: cells immobilization, spore immobilization, reduced cells washout, increased biomass sedimentation, cells recycling, reduced preculture volume, down time reduction, increased titer (g/L), increased conversion (g substrate/g products), increased productivity (g/(L/h)),

Example of applications and benefits for enzymes immobilization are: enzymes immobilization, convert batch process to continuous process, enzymes re-uses for multiples batches, increased enzymes stability, reduced enzyme consumption cost, enzymes recycling, reduced enzyme washout, etc.

### Use of Silica Microcapsules and Silica-Carbon Allotrope Composite Microparticles as Adsorbents for Analyte or Toxic Molecules

According to another embodiment, due to their high surface area and their chemical functionalization, silica microcapsules and their corresponding silica-carbon allotrope microparticles of the present invention can be used as excellent adsorbents for different chemical and biological species. The mentioned species can be polar or non-polar pollutants present in water or in air (e.g. heavy metals, sulphates, phosphates, phenols, dyes, aromatics, hydrocarbons, halogenated organic compounds, proteins, H₂S, etc.)

### Use of Silica-Carbon Allotrope Microparticles as a Sporulation Inducer

According to an embodiment, in certain conditions and depending on the surface chemistry of the carbon allotrope moiety, silica-carbon allotrope microparticles may be used as a sporulation inducer instead of an immobilization carrier. The sporulation inducing properties can be used in biological applications such as, but not limited to, industrial fermentation, food industry, environmental biotechnology, etc.

Silica-carbon allotrope composite microparticles of the present invention used for sporulation are suitable for biological reactor such as, but not limited to, fermentation batch reactor, membrane bioreactor (MBR), moving bed bioreactor (MBBR), fluid bed reactor (FBR), continuous stirred reactor (CSTR), plug flow reactor (PFR), etc. Reactor and bioprocess can be run under anaerobic and aerobic conditions. Silica carbon allotrope composite of the present invention can be added to a reactor at any moment before, during or after fermentation.

The present invention will be more readily understood by referring to the following examples which are given to illustrate the invention rather than to limit its scope.

### EXAMPLE 1

### Chemical Coating of Graphene Oxide on Silica Microcapsules

Prior to use, graphene oxide (GO) was produced from graphite flakes using a modified Hummers method (Hummers, W. and Offeman, R.; J. Am. Chem. Soc. 1958, 80, 1339). Amino-functionalized silica microcapsules were produced according to International patent Application publication No. WO2013/078551.

In a first step, 2 g of GO was dispersed by ultrasonication in 500 mL of DMF, followed by the addition of 9 g of amino-functionalized silica microcapsules and 2 g of DCC (N,N′-dicyclohexyl carbodiimide). The mixture was then stirred at 50° C. for 18 hours before being washed several times with water and methanol in order to remove the unbound GO, and finally dried to obtain a grey powder.

### EXAMPLE 2

### In Situ Chemical Synthesis of Silica Nanoparticles on the Surface of Graphene Sheets

Prior to use, graphene oxide (GO) was produced from graphite flakes using a modified Hummers method (Hummers, W. and Offeman, R.; J. Am. Chem. Soc. 1958, 80, 1339).

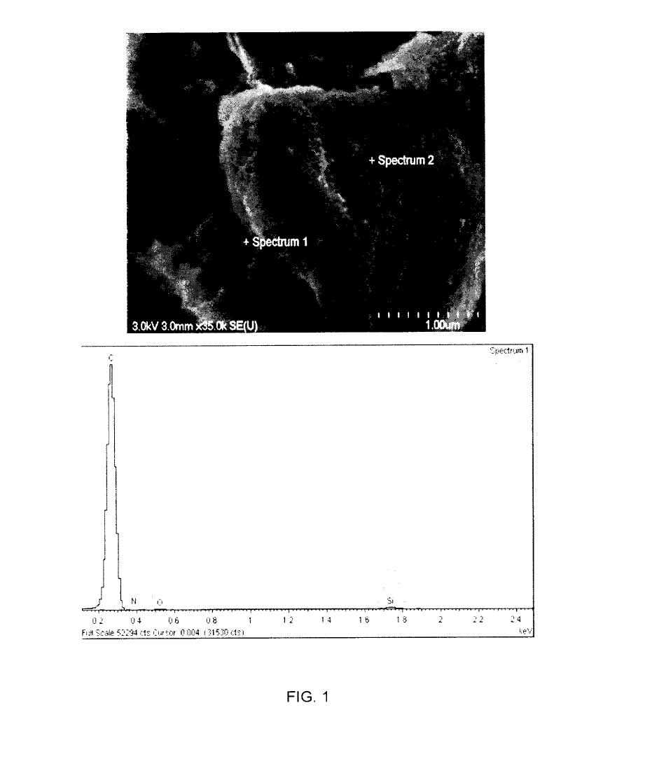

1 g of GO and 17 g of TEOS were dispersed separately in 150 mL of ethanol. The obtained stable suspensions were mixed together and stirred at 40° C. for 15 min. In a next step, 2.5 g of an ammonia solution (28% w/w) was added into the previous mixture and stirred at 40° C. for 20 hours. The resulting product was washed several times with water and ethanol and finally dried to yield a grey powder. SEM image and the corresponding spectra of graphene flakes covered with silica nanoparticles are shown in FIG. 1.

### EXAMPLE 3

### Synthesis of Graphene Using Plasma Deposition Process

Before the step of the production of silica-graphene composite materials, graphene was synthesized alone using the plasma deposition process (Scheme 1), according to a previously reported method (Plasma Chem. Plasma Process (2011) 31:393-403).

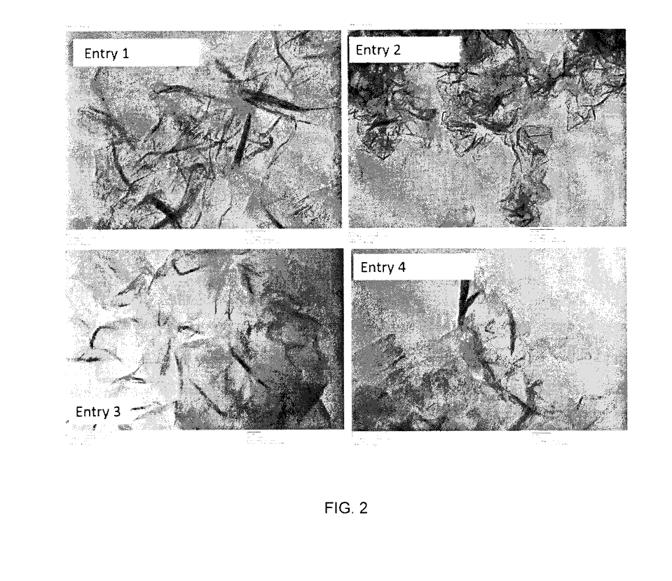

In this process, the plasma is produced using an inductively coupled radio-frequency torch operated at powders ranging from 8 to 20 kW.). In typical experiments, methane was chosen to be used as the carbon source and the central plasmagenic gas, while argon was used as the sheath gas. The carrier gas was composed of methane diluted in argon at different concentrations ranging from 1.7 to 8% v/v. Detailed operating parameters used for the synthesis of graphene via the plasma deposition process are described in Table 1 and representative graphene TEM images are shown in FIG. 2.

TABLE 1
Operating parameters used for the synthesis of graphene via the plasma
deposition process

| | | | | | | Central/ | | |
|---|---|---|---|---|---|---|---|---|
| | | | | | Sheath | plasmagen | Probe/ | Run |
| | Power | Pressure | CH₄/ | CH₄ | gas | gas | carrier gas | time |
| Entry | (kW) | (kPa) | (CH₄ + Ar) | inlet port | (slpm) | (slpm) | (slpm) | (min) |
| 1 | 8.2 | 14.00 | 4.5 | Central | 12-Ar | CH₄ | — | 180 |
| 2 | 11.9 | 13.33 | 7.9 | Central | 6-Ar | CH₄ | — | 40 |
| 3 | 15 | 13.33 | 5.1 | Central | 10-Ar | CH₄ | — | 40 |
| 4 | 12.1 | 13.33 | 3.7 | Central | 10-Ar | CH₄ + Ar | — | 45 |
| 5 | 13.8 | 24.00 | 4.5 | Probe | 12-Ar | 0.5-Ar | CH₄ | 35 |
| 6 | 20.2 | 61.33 | 1.7 | Probe | 35-Ar | 0.5-Ar | CH₄ | 20 |

### EXAMPLE 4

### In Situ Formation Graphene onto the Surface of Silica Microcapsules Using Plasma Deposition

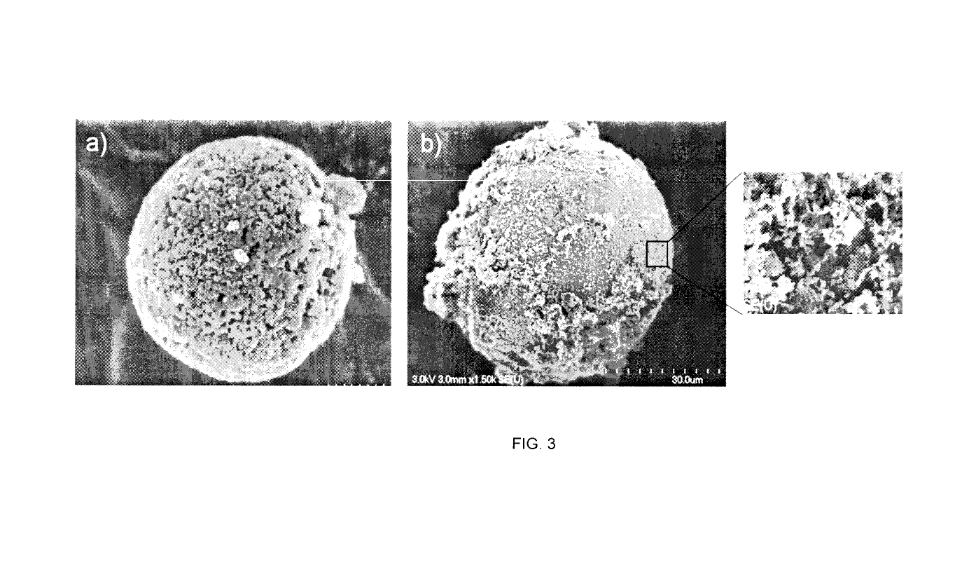

Prior to use, silica microcapsules were produced as described in International Patent Application publication No. WO2013/078551. The suspension of silica microcapsules (typical concentrations of 4-7% wt. microparticles in a solvent that is preferably pure heptane or a water:heptane mixture) is injected using a peristaltic pump in the chamber. Synthesis of carbon allotropes and subsequent in situ deposition on microparticles take place in a chamber operated between 13.33 kPa and 80.00 kPa (100 and 600 Torr). The deposition of the carbon allotropes on the silica microparticles occur in a reactor by injecting a suspension in the vicinity of where the carbon allotrope is formed. Three configurations are possible for the in situ deposition of carbon allotropes on silica microparticles (Scheme 2). The first configuration consists of a main and an auxiliary tubular reactor in which injection is carried out in the probe, and injected concentric to the plasma torch. In a second configuration, the suspension of microparticles is injected through the top flange of the main reactor and is allowed to partly interact with the skirt of the torch. In the third configuration, the suspension of microparticles is injected from the bottom flange and into the periphery of the plume, at the bottom part of the main reactor. Detailed operating parameters used for these experiments are described in Table 2 and representative SEM image of the obtained silica-graphene composite material is shown in FIG. 3.

TABLE 2
Operating parameters used for the deposition of graphene onto the
surface of silica microparticles via the plasma deposition process

| | | | | | | | |
|---|---|---|---|---|---|---|---|
| | | | | | Central/ | Probe/ | |
| | | Pres- | CH₄/ | Sheath | plasmagen | carrier | Run |
| | Power | sure | (CH₄ + | gas | gas | gas | time |
| Entry | (kW) | (kPa) | Ar) | (slpm) | (slpm) | (slpm) | (min) |

Configuration 1

| | | | | | | | |
|---|---|---|---|---|---|---|---|
| 1 | 11.5 | 24.00 | 4.4 | 12-Ar | 0.5-Ar | CH₄ | 15 |
| 2 | 10.9 | 40.00 | 3 | 25-Ar | 0.5-Ar | CH₄ | 15 |
| 3 | 10.9 | 42.66 | 3.4 | 22.5-Ar | 0.5-Ar | CH₄ | 10 |

Configuration 2

| | | | | | | | |
|---|---|---|---|---|---|---|---|
| 4 | 12.4 | 26.66 | 4.6 | 12-Ar | 0.5-Ar | CH₄ | 5 |
| 5 | 12.9 | 30.66 | 4.7 | 8-Ar | 0.1-Ar | CH₄ | 5 |

Configuration 3

| | | | | | | | |
|---|---|---|---|---|---|---|---|
| 6 | 12.9 | 30.66 | 4.7 | 8-Ar | 0.1-Ar | CH₄ | 6 |

### EXAMPLE 5

### In Situ Formation and Functionalization of Graphene onto the Surface of Silica Microcapsules Using Plasma Deposition Process: Doping with Nitrogen-Containing Functional Groups

Prior to use, silica microcapsules were produced as described in International Patent Application publication No. WO2013/078551. In addition to the setup described in Example 4 of the present invention, nitrogen precursors were co-injected using a plasma probe with methane. Methane and ammonia the nitrogen precursor (NH₃, entry 1, Table 3) were injected in the reactor at a ratio of 8CH₄:5NH₃. When N₂ is used as a precursor, a ratio of 16CH₄:17N₂:10H₂ was used. H₂ was added to facilitate the decomposition of N₂ and the subsequent formation of the nitrogen functional group on the graphitic structure. The suspension of silica microcapsules (typical concentrations of 4-7% wt. microparticles in a solvent that is preferably pure heptane or a water:heptane mixture) is injected using a peristaltic pump through the bottom inlet of the chamber (configuration 3) and sprayed in the reactor using an Ar carrier gas. The operating parameters are listed in Table 3.

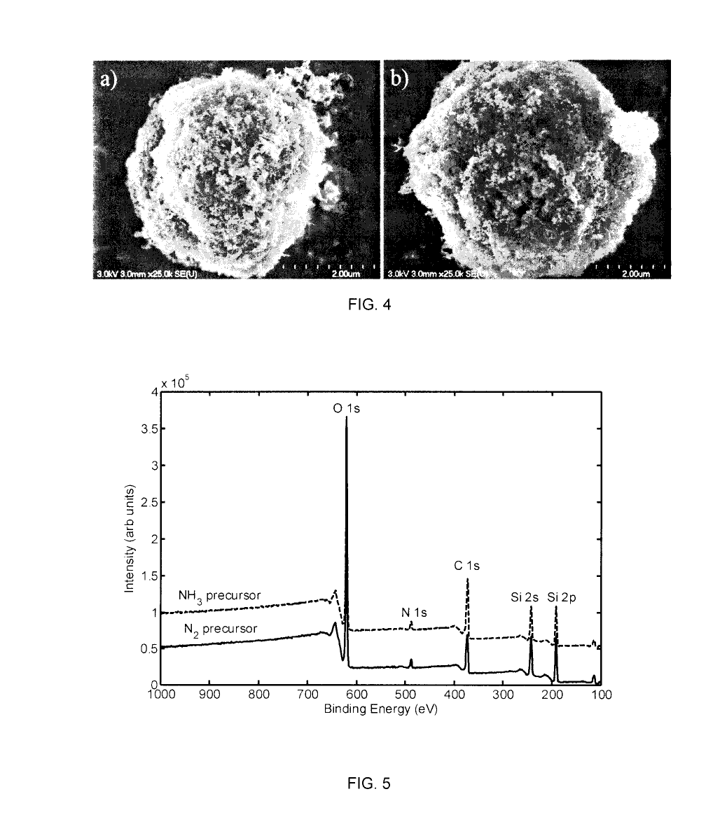

The powders were collected on the walls of the reactor, in the auxiliary reactor and on the filters. Representative scanning electron microscopy (SEM) micrographs of the silica microspheres-functionalized graphene composite show a uniform coverage of the microsphere with carbon nanoplatelets for both NH₃ and N₂ as nitrogen precursors (FIG. 4). In all cases, the SEM observations showed no sign of degradation, melting or collapsing of the microcapsules. The samples produced using the parameters of Table 3 were probed using X-ray photoelectron spectroscopy.

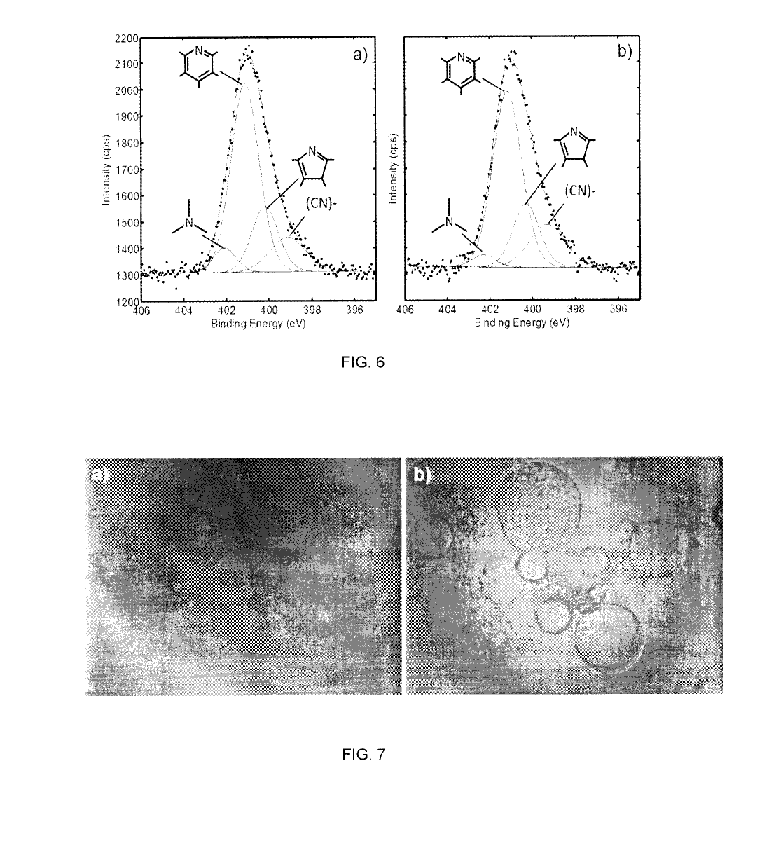

The spectra surveys are shown in FIG. 5 which confirms the presence of nitrogen (N 1s peak at 399 eV), carbon (C 1s peaks at 284.7 eV) and silicon (Si 2p at 130.3 eV and Si 2s at 149 eV) for samples produced using nitrogen precursors. From the XPS survey, the nitrogen content with respect to carbon is estimated to 2.5 at. % and 2.3 at. % when using NH₃ and N₂, respectively. The high resolution spectra of the N 1s peak from samples produced following the parameters described in entries 1 and 2 (Table 3) are shown in FIGS. 6. Fitting of the N 1s peak highlights the presence of various forms of nitrogen bonds to the graphene matrix, including cyanide (399.2 eV), pyrrolic (400.2 eV), pyridinic (401.1 eV) and quaternary (402.3 eV).

TABLE 3
RF plasma parameters during the deposition of functionalized graphene onto
silica microparticles (RT = run time)
Setup: Configuration 3 (Scheme 2)

| | | | | | | | Silica | | |
|---|---|---|---|---|---|---|---|---|---|
| | | | | | | | suspension | | |
| | | | | | | Central/ | spraying | | |
| | | | | Probe/reactant | Sheath | plasmagen | | Carrier | |
| | | | | gas ratio | gas | gas | | gas | |
| | | Power | Pressure | Molar flow | Ar | Ar | Feed | Ar | RT |
| Entry | Samples | (kW) | (kPa) | ratio | (slpm) | (slpm) | mL/min | (slpm) | (min |
| 1 | graphene- | 19.4 | 80 | 8 CH₄:5 NH₃ | 42 | 2 | 6.5 | 20 | 10 |
| | NH₃/Silica | | | | | | | | |
| 2 | graphene- | 19.6 | 80 | 16 CH₄:17 | 42 | 2 | 6.5 | 20 | 10 |
| | N₂/Silica | | | N₂:10 H₂ | | | | | |

### EXAMPLE 6

### Silica Microcapsules and Silica-Graphene Microparticles Used as Adsorbents for Chemical or Biological Species

For adsorption experiments, 50 mg of silica microcapsules produced as described in International Patent Application publication No. WO2013/078551 or silica-graphene microparticles of the present invention were mixed with solutions containing 50 mg of different chemical or biological species including farnesol (terpene), catechol (polyphenol), butyric acid, vaniline, glucose, furfural and proteins (Bovine Serum Albumine). After 5 minutes of stirring, the obtained mixtures were centrifuged and the supernatants were analyzed using High-Performance Liquid Chromatography (HPLC). The results summarized in Table 4 show very high adsorption rates (from 250 to 750 mg/g) depending on the type of molecules and adsorbents.

TABLE 4
Adsorption performances of silica microcapsules produced as described
in International Patent Application publication No. WO2013/078551
and silica-graphene microparticles of the present invention

| Compound | Adsorbent | Adsorption rate (mg/g) |
|---|---|---|
| Terpene | Silica-Graphene | 258 |
| (Farnesol) | microparticles | |
| Polyphenol | Silica microcapsules | 340 |
| (Catechol) | | |
| Butyric acid | Silica microcapsules | 405 |
| Vaniline | Silica microcapsules | 355 |
| Glucose | Silica microcapsules | 312 |
| Furfural | Silica microcapsules | 299 |
| Phosphate | Silica microcapsules | 400 |
| Ammonia | Silica microcapsules | 310 |
| Proteins | silica microcapsules | 721 |
| (Bovine Serum | | |
| Albumine) | | |

### EXAMPLE 7

### Silica Microcapsules as a Carrier for Bacteria Immobilization

In order to demonstrate the use of silica microcapsules as carriers for the immobilization of bacteria, several experiments have been performed taking into account the presence or not of silica microcapsules and the use or not of a LB medium (a nutritionally rich medium). Prior to use, the LB medium was prepared by adding 10 g of tryptone, 5 g of yeast extract and 10 g of NaCl in 1 L of water, and the mixture was sterilized in an autoclave. Peptone water, which is a control medium, was prepared by adding 9 g of NaCl and 1 g of peptone in 1 L of water, and then sterilized in an autoclave. Silica microcapsules were produced according to International patent application publication No. WO2013/078551 as slurry containing 7.4% w/w of silica in water.

### Bacteria in Peptone Water Without Silica Microcapsules

25 μL of Bacillus subtilis stored at −80° C. in 30% glycerol was added in 100 mL of peptone water and incubated at 37° C. under stirring. After 24 hours, a sample of 500 μL was then taken and observed by optical microscopy (FIG. 7a). Any biofilm formation is observed on this picture.

### Bacteria in Peptone Water in the Presence of Silica Microcapsules

4.25 g of silica microcapsules slurry was prewashed with peptone water according to the following steps. A solution containing silica microcapsules and a given volume of peptone water was centrifuged for 10 minutes at 5000 g. This washing step was performed twice, followed by a sterilization step in an autoclave. The resulting solution was centrifuged again for 10 minutes at 5000 g and the supernatant was taken in sterile conditions. In a next step, the obtained silica microcapsules were dispersed in 100 mL of peptone water. 25 μL of Bacillus subtilis was then added to 100 mL of the resulting silica microcapsule solution and incubated at 37° C. under stirring. After 24 hours, a sample of 500 μL was taken and observed by optical microscopy (FIG. 7b). This picture clearly shows the immobilization of bacteria on the surface of silica microcapsules and the formation of biofilm.

### Bacteria in LB Medium in the Presence of Silica Microcapsules

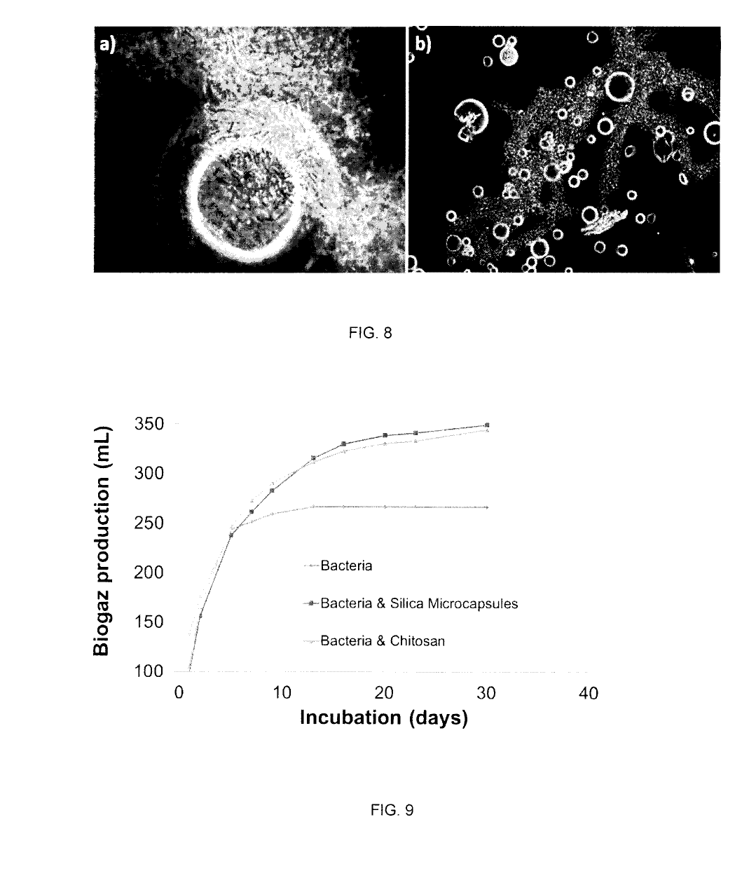

4.25 g of silica microcapsules slurry was prewashed with LB medium according to the following steps. A solution containing silica microcapsules and a given volume of LB water was centrifuged for 10 minutes at 5000 g. This washing step was performed twice, followed by a sterilization step in an autoclave. The resulting solution was centrifuged again for 10 minutes at 5000 g and the supernatant was taken in sterile conditions. In a next step, the obtained silica microcapsules were dispersed in 100 mL of peptone water. Then, 25 μL of Bacillus subtilis was added to this solution and incubated at 37° C. under stirring. After 24 hours, a sample of 500 μL was taken and observed by optical microscopy (FIG. 8). On these images, a dense biofilm with long branches was formed on silica microcapsules.

### EXAMPLE 8

### Silica Microcapsules as a Carrier of Microoganism for Increased Methane Production

In order to evaluate silica microcapsule potential for increased methane production under anaerobic condition, silica microcapsule were added to wastewater with microorganisms in lab scale experiments to test for biochemical methane potential. The experiment was done using synthetic wastewater.

The synthetic waste water is composition is: 630 mg/L glucose, 220 mg/L powdered milk, 14 mg/L glutamic acid, 80 mg/L ammonium sulfate, 5 ammonium chloride, 10 mg/L magnesium sulfate, 3 mg/L manganese sulfate, 3 mg/L calcium chloride, 0.3 mg/L ferric chloride, 14 mg/L potassium phosphate (monobasic), 28 mg/L potassium phosphate (dibasic).

The microorganisms used are from flocs from an upflow anaerobic sludge blanket (UASB) reactor. Flocs are crushed before being used as an inoculum.

Experiments were done in 250 ml flask with 125 ml working volume. The flasks are purge every 2 minute with N₂/CO₂ (80% N₂, 20% CO₂). The experiment is done at 37° C. under 200 rpm over 25 days. . Five grams of UASB microorganisms are used as an inoculum for each tested conditions.

Three condition are evaluated. The first consist of UASB microorganisms in the synthetic waste water without microcapsule, the second is the UASB microorganisms in the synthetic waste water with 1 g/L silica microcapsule and the third is the UASB microorganisms in the synthetic waste water with 1 g/L chitosan. Each conditions are done in triplicate.

Cumulative methane production from time zero to day 30 is show in FIG. 9. This figure shows that after 30 days, microorganisms in combination with silica microcapsule produced 30% more methane than microorganisms without silica microcapsule.

### EXAMPLE 9

### Silica Microcapsules as a Carrier For Bacteria in Order to Increase Biomolecule Production in Pilot Bioreactor

In order to demonstrate the potential for increased biomolecules production, a fermentation of Bacillus licheniformis producing protease was done in presence of silica microcapsules.

Three conditions were tested. The first is the control (no microcapsule). The second is a high microcapsule condition (3 g/L). The third is a low microcapsule solution (0.6 g/L)

The culture nutrient broth was as follow: 14.9 g/L of soy hydrolysate, 11.36 g/L of Na₂HPO₄, 9.6 g/L of NaH₂PO₄, 0.16 g/L MgSO₄ heptahydrate, 0.374 g/L of CaCl₂ dihydrate and 48 g/L of glucose. The pH was adjusted to 7.5 after bacteria addition.

Microcapsule are introduced in the preculture. Microcapsule and glucose are prepared together separately from the rest of the nutrient broth and added later to the preparation. The preculture is incubated at 37° C. for 24 h at 250 rpm.

The 1 L bioreactors are first inoculated with a 60 ml preculture. Bioreactor condition are: 37° C., no pH control, aeration of 1 L/min, 300 to 650 rpm of agitation depending on oxygen demand.

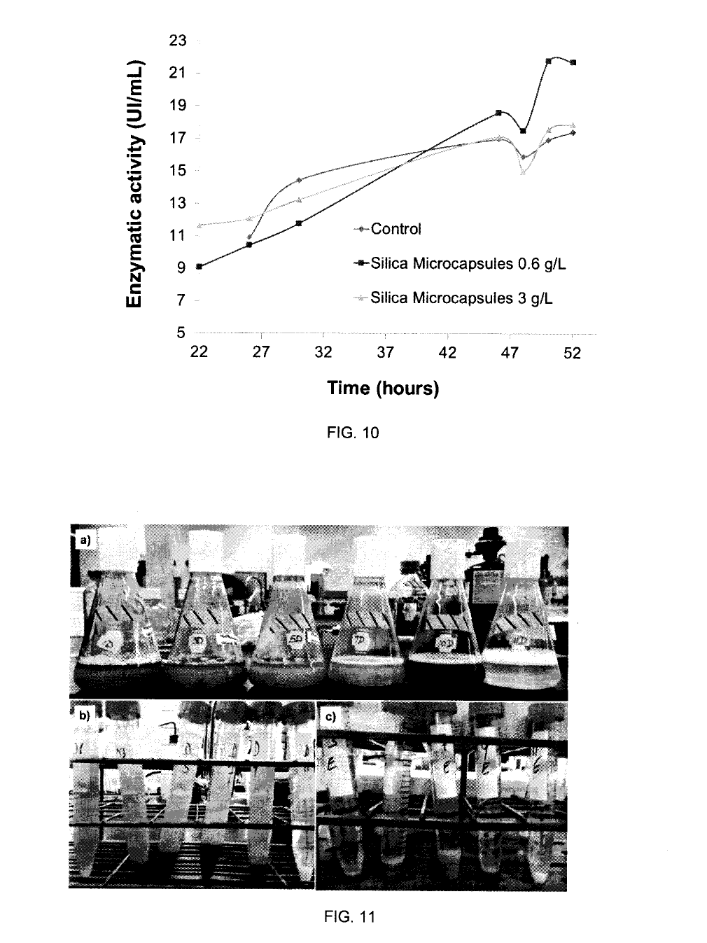

Sample are taken at 22, 26, 30, 46, 48, 50 and 52 hour from the bioreactor and use to determine the enzymatic activity of the protease produced from the bacteria. The enzymatic activity determination will be used as an indirect measure of enzymes production. Enzymatic activity is quantified using Sigma Aldrich method for protease enzymatic activity quantification. Enzymatic activity of the three different conditions are show in FIG. 10.

In FIG. 10 it is shown that 0.6 g/L yield more enzyme than 3 g/L. Previous results has shown that silica microcapsules benefits are lost when using too much microcapsule since cells are detached by high shear stress generated by a high particle concentration. At 0.6 g/L, the enzymatic activity is approximately 25% higher using the silica microcapsules compared to fermentation without microcapsule. Although conditions are not optimized, result provide a clear demonstration of the potential for increased biomolecules production using silica microcapsules.

### EXAMPLE 10

### Silica Microcapsules as a Carrier for Yeast Immobilization and Qualitative Demonstration of Adhesion Strength

Similar to example 6, microorganisms were growth in a growth media using silica microcapsules. Instead of using a bacteria, a yeast was used (saccharomyces cerevisiae).

Sample number 1 consists of yeasts without microcapsules. Sample 2 to sample 4 consist of yeast with increasing concentration of microcapsules. Sample 5 is the growth media with microcapsules but without yeast. Sample 6 consist of microcapsules in water.

After 48 hours of incubation, 10 ml of each sample is transferred to 15 ml falcon tube. Samples are then let sill for 30 minutes at room temperature in order for sedimentation to occur. Supernatant is taken out and the sample is then washed with saline (0.9% NaCl) in order to evaluate if cells can be detached. Washing is done by vigorous tube inversion.

A picture is taken right after incubation (FIG. 11a), after sedimentation (FIG. 11b) and after washing (FIG. 11c) for qualitative analysis. Sample number 1 is not in FIG. 11c since it cannot be washed because sedimentation could not occur since the sample did not contain microcapsules.

Starting from sample number 1 to sample number 4, it can be seen that the culture broth change color from brown to light brown indicative of an increased biomass density (FIG. 11a). This suppose that increased microcapsule concentration gave rise to higher biomass density. Sample number 6 shows that the color change does not come from the microcapsules.

FIG. 11b illustrates that the microcapsule has been separated from the supernatant by gravity and it confirms that microcapsules has a good potential for gravity separation.

FIG. 11c shows that the washing solution is clear and a clear distinction is made between the microcapsule and the washing solution. It suggest that the microcapsule strongly bind the both the cells and the culture medium pigment.

### EXAMPLE 11

Silica-Carbon Allotrope Composite Microparticles Used as a Sporulation Inducer

In order to demonstrate the use of silica-carbon allotrope composite microparticles as sporulation inducers, Bacillus subtilis was grown in peptone water. Two bacterial preparations were made and contained the same ingredients, except for the fact that one preparation contained silica-carbon allotrope composite microparticles. The bacterial preparation without microparticle is defined as the positive control. The experiment also contained a preparation without bacteria and without silica-carbon allotrope composite microparticles, which are defined as the negative controls.

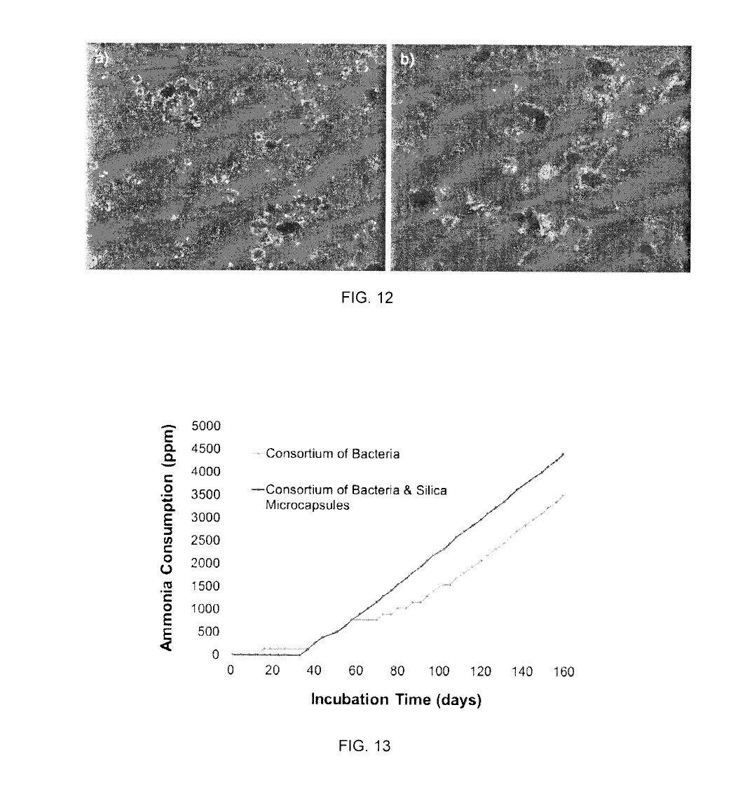

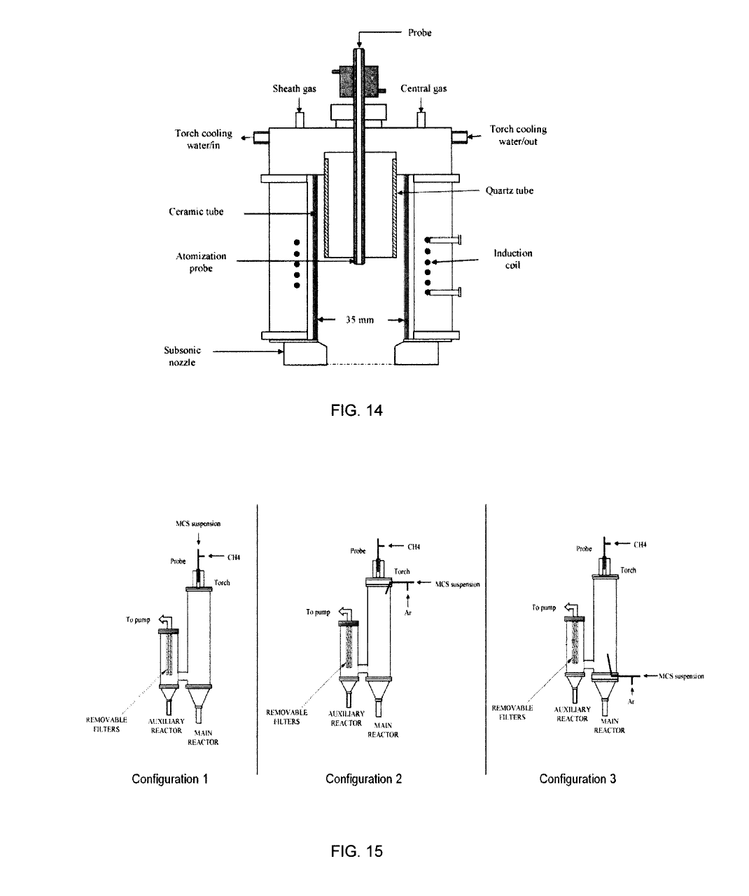

The peptone water contained 9 g/L of NaCl and 1 g/L of peptone. The microparticles were used at a concentration of 2.5 g/L. Bacillus subtilis inoculum was kept in 30% glycerol at −80° C. The bacterial preparations consisted of 25 μl of inoculum added to 100 ml of peptone water. The experiment took place in 500 ml sterile Erlenmeyer flasks under 200 round per minutes (rpm) agitation at 37° C. The incubation lasted 24 hours. Sporulation evaluation was done with optical microscopy at 100 and 1000× (FIG. 12).

Optical microscopy observation showed that bacterial preparation with microcapsule contained spores. The bacterial preparation without microcapsule, the positive control, did contain bacteria but did not contain spores. No growth were observed in the negative controls.

### EXAMPLE 12

### Silica Microcapsules as a Carrier for Alpha-Amylase Immobilization

For enzyme immobilization experiments, amylase (from Bacillus Licheniformis) was added at a concentration of 1 unit/mL in a buffered solution containing 20 mM of Sodium Phosphate and 6.7 mM of Sodium Chloride at pH 6.9. To this solution, silica microcapsules produced as described in International Patent Application publication No. WO2013/078551 were added at a concentration of 2.5 mg/mL and then agitated for 5 minutes. Enzymes are immobilized to silica microcapsules by adsorption which occur naturally.

The standard method used to determine the enzyme activity was obtained from the enzyme supplier (Sigma Aldrich). The Sigma Aldrich's method is named enzymatic assay of a-amylase and it is based on P. Bernfeld methods (Methods in Enzymology, 1955). The enzymatic activity of both free and immobilized enzyme was evaluated at pH 7 at a temperature of 20° C. This was compared to a control enzyme solution without silica microcapsules. Results show a mean enzyme immobilization efficiency 95% calculated from 5 replicates. The immobilization efficiency was defined as the immobilized enzymes activity over the free enzymes activity.

### EXAMPLE 13

### Silica Microcapsules as a Carrier for Glucose Oxidase Immobilization

Similarly to example 12, the enzyme a glucose oxidase that produces hydrogen peroxide, was immobilized on silica microcapsule using similar condition.

In example 10, immobilization was done by simple adsorption. In this example, immobilization is done by adsorption and is made more robust by adding varying solutions of glutaraldehyde (20 to 1000 mmol/L). In this example, enzymes stability is challenged. The glucose oxidase produces hydrogen peroxide which is detrimental to enzymes function.

The best immobilization conditions gave an immobilization efficiency of 123%. The immobilization efficiency was defined as the immobilized enzymes activity over the free enzymes activity. For all conditions, the immobilized enzymes were more productive than the free enzyme. Increased productivity of immobilized enzymes is due to increased stability provided by immobilization in silica micro particles pores. Benefits of enzymes immobilization such as increased stability is well defined in the scientific literatures.

### EXAMPLE 14

### Silica Microcapsules Used as a Carrier for Bacteria in Order to Increase Nitrification

To evaluate silica microcapsule potential for increased nitrification reactor production under aerobic condition, silica microcapsule were added to waste water in lab scale experiments to evaluate consumption of ammonia. The microorganisms used were a nitrification consortium. The experiment was done using synthetic waste water.

The experiment was done in 250 ml flask with 125 ml working volume. The experiment is done at room temperature at 115 rpm over a 160 days period. Potassium carbonate is added to maintain a stable pH.

Two conditions were evaluated. The first consist of a consortia in the synthetic waste water without silica microcapsule, the second is the consortium in synthetic waste water with 1 g/L silica microcapsule.

Cumulative ammonia consumption from time zero to day 160 is shown in FIG. 13. The figure shows that the consortia without microcapsule has an inconsistent ammonia consumption rate. On the other hand, using silica microcapsule, the ammonia cumulative consumption is steady and the total ammonia consumed is significantly greater by 25 to 65% from day 90 to day 160.

While preferred embodiments have been described above and illustrated in the accompanying drawings, it will be evident to those skilled in the art that modifications may be made without departing from this disclosure. Such modifications are considered as possible variants comprised in the scope of the disclosure.

1. A carbon allotrope-silica composite material comprising:
a silica microcapsule comprising:
a silica shell having a thickness of from about 50 nm to about 500 μm, and a plurality of pores,
said shell forming a capsule having a diameter from about 0.2 μm to about 1500 μm, and having a density of about 0.001 g/cm³ to about 1.0 g/cm³,
wherein said shell comprises from about 0% to about 70% Q3 configuration, and from about 30% to about 100% Q4 configuration, or
wherein said shell comprises from about 0% to about 60% T2 configuration and from about 40% to about 100% T3 configuration, or
wherein said shell comprises a combination of T and Q configurations thereof, and
wherein an exterior surface of said capsule is covered by a functional group;
a carbon allotrope attached to said silica microcapsule.

2. A carbon allotrope-silica composite material comprising:
a carbon allotrope attached to a silica moiety comprising a silica nanoparticle having a diameter from about 5 nm to about 1000 nm, wherein an exterior surface of said silica nanoparticle is covered by a functional group.

3. The carbon allotrope-silica composite material of claim 1, wherein said thickness of said silica microcapsule is from about 50 nm to about 240 μm.

4. The carbon allotrope-silica composite material of claim 1, wherein said diameter of said silica microcapsule is from about 0.2 μm to about 500 μm.

5. The carbon allotrope-silica composite material of claim 1, wherein said density of said silica microcapsule is from about 0.01 g/cm³ to about 0.5 g/cm³.

6. The carbon allotrope-silica composite material of claim 1, wherein said carbon allotrope is attached covalently to said functional group of said silica particle.

7. The carbon allotrope-silica composite material of claim 1, wherein said carbon allotrope is attached non-covalently to the surface of said silica particle.

8. The carbon allotrope-silica composite material of claim 1, wherein said functional group of said silica particle is a hydroxyl group, a carboxylic acid group, a thiol group, an amino group, a benzylamino group, a chloropropyl group, a disulfide group, an epoxy group, a mercapto group, a methacrylate group, a vinyl group, and combinations thereof.

9. The carbon allotrope-silica composite material of claim 1, wherein said carbon allotrope is functionalized or not functionalized.

10. The carbon allotrope-silica composite material of claim 1, wherein said functional group of said carbons allotrope is a nitrogen-containing functional group, an oxygen containing functional group, a sulfur-containing functional group, a halogen-containing functional group and a combination thereof.

11. The carbon allotrope-silica composite material of claim 10, wherein said nitrogen-containing functional group is an amine group, a ketimine group, an aldimine group, an imide group, an azide group, an azo group, a cyanate group, an isocyanate group, a nitrate group, a nitrile group, a nitrite group, a nitroso group, a nitro group, a pyridyl group and a combination thereof, and
wherein said sulfur-containing functional group is an sulfhydryl group, a sulfide group, a disulfide group, a sulfinyl group, a sulfonyl group, a sulfo group, a thiocyanate group, carbonothioyl group, carbonothioyl group and a combination thereof, and
wherein said sulfur-containing functional group is an sulfhydryl group, a sulfide group, a an aldehyde group, a carboxylate group, a carboxyl group, an ester group, a methoxy group, a peroxy group, an ether group, a carbonate ester and a combination thereof, and
wherein said halogen-containing functional group is a fluoro, a chloro, a bromo, an iodo and a combination thereof.

12.-14. (canceled)

15. The carbon allotrope-silica composite material of claim 1, wherein said carbon allotrope is chosen from graphite, graphene, a carbon nanofiber, a carbon nanotubes, a C60 fullerene, a C70 fullerene, a C76 fullerene, a C82 fullerene, a C84 fullerene, and a combination thereof.

16. The carbon allotrope-silica composite material of claim 1, wherein said silica shell of said silica microcapsule comprises from about 40% Q3 configuration and about 60% Q4 configuration, or from about 100% Q4 configuration.

17. The carbon allotrope-silica composite material of claim 1, wherein said pores of said silica microcapsule have pore diameters from about 0.5 nm to about 100 nm.

18. The carbon allotrope-silica composite material of claim 1, wherein said functional group of said silica microcapsule is a hydroxyl group, an amino group, a benzylamino group, a chloropropyl group, a disulfide group, an epoxy group, a mercapto group, a methacrylate group, a vinyl group, and combinations thereof.

19. The carbon allotrope-silica composite material of claim 18, wherein said functional group is provided by an organosilane chosen from a functional trimethoxysilane, a functional triethoxysilane, a functional tripropoxysilane, 3-aminopropyltriethoxysilane, vinyltriacetoxy silane, a vinyltrimethoxysilane, 3-glycidoxypropyltrimethoxysilane, 3-methacryloxypropyltrimethoxysilane, 3-chloropropyltriethoxysilane, a bis-(triethoxysilylpropyl)tetrasulfane, a methyltriethoxysilane, a n-octyltriethoxysilane, and a phenyltrimethoxysilane and combinations thereof.

20. The carbon allotrope-silica composite material of claim 1, wherein said carbon allotrope-silica composite material is loaded with a molecule.

21. The carbon allotrope-silica composite material of claim 20, wherein said molecule is a fluorescent molecule, a magnetic particle, a catalyst molecule, a biological macromolecule, or a combination thereof.

22. The carbon allotrope-silica composite material of claim 21, wherein said magnetic molecule is a magnetic nanoparticle.

23.-109. (canceled)
